(19) United States
(12) Patent Application Publication
(10) Pub. No.: US 2026/0272984 A1
(43) Pub. Date: Sep. 17, 2026

(54) MEDIA AND METHODS FOR DIFFERENTIATING T CELLS FROM HEMATOPOIETIC STEM CELLS

(71) Applicant: The Regents of the University of California, Oakland, CA (US)

(72) Inventors: Lili Yang, Los Angeles, CA (US); Derek Lee, Los Angeles, CA (US); Kuangyi Zhou, Los Angeles, CA (US)

(73) Assignee: The Regents of the University of California, Oakland, CA (US)

(21) Appl. No.: 19/474,055

(22) PCT Filed: Mar. 29, 2024
(86) PCT No.: PCT/US24/22157

§ 371 Date: Oct. 9, 2025

Related U.S. Application Data
(60) Provisional application No. 63/497,510, filed on Apr. 21, 2023.

(51) Int. Cl. A61K 35/17; A61K 40/11; A61K 40/31; A61K 40/32; A61K 40/42; A61P 35/00; C07K 14/54; C07K 14/705; C12N 5/00; C12N 5/0783; C12N 15/86

(52) U.S. Cl. CPC A61K 35/17; A61K 40/11; A61K 40/31; A61K 40/32; A61K 40/421; A61K 40/4234; A61P 35/00; C07K 14/5443; C07K 14/7051; C07K 14/70514; C07K 14/70517; C12N 5/0018; C12N 5/0087; C12N 5/0636; C12N 15/86; C12N 2500/25; C12N 2500/38; C12N 2500/44; C12N 2500/99; C12N 2501/125; C12N 2501/145; C12N 2501/2303; C12N 2501/2306; C12N 2501/2307; C12N 2501/2315; C12N 2501/26; C12N 2506/1369; C12N 2506/45; C12N 2510/00; C12N 2740/15043

(57) ABSTRACT

Embodiments of the disclosure include compositions and methods related to differentiate and engineer hematopoietic stem and progenitor cells (HSPCs or HSCs) to mature T cells (Diff-T cells) for off-the-shelf use for clinical therapy. Embodiments of the media are serum-free, and/or the culture methods are feeder-free. The Diff-T cell products can be conventional aP T cells, yS T cells, and unconventional T cells such as invariant natural killer T (iNKT) cells, non-invariant nature killer T (NKT) cells, mucosal-associated invariant T (MAIT) cells, and others. The Diff-T cell products can also be designed to be the CD8-positive or CD4-positive T cell subtypes, and the CD4-positive Diff-T cells can be further designed to be various TH-polarized subtypes.

### CROSS REFERENCE TO RELATED APPLICATIONS

This application claims the benefit under 35 U.S.C. Section 119(e) of co-pending and commonly-assigned U.S. Provisional Patent Application No. 63/497,510, filed Apr. 21, 2023, entitled “MEDIA AND METHODS FOR DIFFERENTIATING T CELLS FROM HEMATOPOIETIC STEM CELLS”, the contents of which is incorporated by reference herein.

### TECHNICAL FIELD

Embodiments of the disclosure concern at least the fields of immunology, cell biology, and medicine, including at least cancer medicine.

### BACKGROUND OF THE INVENTION

Cancer affects tens of millions of people worldwide and is a leading threat to public health in the United States. Despite the existing therapies, cancer patients still suffer from the ineffectiveness of these treatments, their toxicities, and the risk of relapse. Novel therapies for cancer are therefore desperately needed. Over the past decade, immunotherapy has become the new-generation cancer medicine. In particular, cell-based cellular therapies have shown great promise. An outstanding example is the chimeric antigen receptor (CAR)-engineered adoptive T cells therapy, which targets certain blood cancers at impressive efficacy.

However, most of the current protocols for treatment consist of autologous adoptive cell transfer, wherein immune cells collected from a patient are manufactured and used to treat this single patient. Such an approach is costly, manufacture labor intensive, and difficult to broadly deliver to all patients in need. Allogenic immune cellular products that can be manufactured at a large-scale and can be readily distributed to treat a higher number of patients therefore are in great demand.

Research into the ex vivo differentiation of T cells from stem cells has been getting more attention in recent years because it has shown its value in understanding T cell development and in generation of off-the-shelf, allogeneic cell products for the treatment of various diseases such as cancer. Previous research has demonstrated that conventional αβ T cells can be differentiated from hematopoietic stem cells (HSCs) or induced pluripotent stem cells (iPSCs) by co-culturing the stem cells with DLL1 or DLL4 expressed OP9 or MS5 feeder cells¹⁻⁴. In more recent years, several studies reported the use of feeder-free and serum-free ex vivo culture to generate T cells⁵⁻⁸. However, according to the data reported, numerous systems produced CD3+ T cells at lower frequencies, maturity levels, and yields compared to those cultures dependent on feeders. Furthermore, these prior systems predominantly generated CD8 single positive cytotoxic T cells, with minimal to no success in producing other T cell subsets, including CD4 single positive helper T cells, as well as innate T cell types such as γδ T cells, natural killer T (NKT) cells, and mucosal-associated invariant T (MAIT) cells.

Novel methods and materials useful in therapies for diseases such as cancer and autoimmune diseases, are in desperate demand. The present disclosure provides solutions to one long-felt need by providing materials and methods useful in immunotherapies that can then be delivered or distributed more widely than the conventional alternatives.

### SUMMARY OF THE INVENTION

The disclosure presented herein provides feeder-free/serum-free ex vivo culture methods and systems that are able to differentiate a variety of T cell lineages from hematopoietic stem cell progenitors. The T cell differentiation systems disclosed herein are termed herein “Diff-T”. As discussed below, the Diff-T culture system comprises Diff-T media and Diff-T methods designed to allow the the effective differentiation of human HSCs into selected lineages of functional human T cells including CD8 single positive cytotoxic T cells, CD4 single positive helper T cells, γδ T cells, Natural Killer T cells, Mucosal-associated invariant T cells, and the like.

Embodiments of the invention comprise new medias (termed as Diff-T medias) and methods for using these medias (termed as Diff-T methods) to differentiate human T cells (Diff-T cells) from hematopoietic stem cell progenitors. Typically, the Diff-T medias are serum-free, and the Diff-T culture methods are feeder-free. As discussed below, embodiments of the invention can be used to generate a variety of different kinds of differentiated T cell lineages from hematopoietic stem cells in high yield, with the resultant cells having robust qualities. In illustrative embodiments of the invention, HSCs are differentiated into various types of T cells, including γδ T cells (e.g., Vδ1 and Vδ2 T cells), conventional αβ T cells (e.g., CD4 helper T cells, CD8 cytotoxic T cells), and unconventional of T cells (e.g., natural killer T cells, mucosal associate-invariant T cells), without gene engineering. In other illustrative embodiments, purified T cells carrying exogenous polynucleotides such as those encoding antigen specific TCRs (e.g., Vδ2 T cells, MAIT cells, iNKT cells) can be generated through TCR gene-engineering of CD34+ HSCs, followed by their ex vivo differentiation into transgenic T cells. In some embodiments of the invention, HSCs are differentiated into mature CD8 single positive (SP) Diff-T cells in a feeder-free/serum-free ex vivo culture. In other embodiments of the invention, HSCs are differentiated into mature CD4 SP Diff-T cells in a feeder-free/serum-free ex vivo culture. In embodiments of the invention, mature Diff-T cells can be further expanded in an ex vivo culture that is either feeder-free or feeder-dependent (e.g., using natural or artificial antigen presenting cells).

In embodiments of the invention, the Diff-T cell culture methods can be combined with HLA-I/II gene-editing and HLA-C/HLA-E/HLA-G/CD47 gene-engineering technologies to produce HLA-I/II-negative HLA-C/HLA-E/HLA-G/CD47-positive Universal Diff-T (^{U}Diff-T) cells, that are suitable for allogeneic adoptive transfer and therefore can be utilized as off-the-shelf cellular product. In embodiments of the invention, the Diff-T cell culture can be combined with RAG-I/RAG-II gene-editing to create RAG-negative Universal TARGET (^{U}Diff-T) cells. These cells prevent endogenous TCR recombination and are amenable to engineering with monoclonal transgenic TCRs. In addition to the antigen-specificity endowed by the monoclonal transgenic TCR, ^{U}Diff-T cells can be further engineered to express additional targeting molecules to enhance their disease-targeting capacity. Such targeting molecules can be Chimeric Antigen Receptors (CARs), other T cell receptors (TCRs), CD16 (FCδRIII), natural or synthetic receptors/ligands, or others. The resulting ^{U}CAR-Diff-T cells, ^{U}TCR-Diff-T cells, or ^{U}X-Diff-T cells can then be utilized for off-the-shelf disease-targeting cellular therapy. In embodiments of the invention, the Diff-T cells and derivatives can also be further engineered to overexpress genes encoding T cell stimulatory factors, or to disrupt genes encoding T cell inhibitory factors, resulting in functionally enhanced Diff-T cells and derivatives. Selected monoclonal TCR genes can encode, for example, a conventional αβ TCR (a CD4 TCR or a CD8 TCR), an invariant NKT (iNKT) TCR, a non-invariant NKT TCR, a MAIT TCR, a γδ TCR, or the like.

T cells generated by embodiments of the invention disclosed herein can be utilized in a variety of therapeutic regimens such as those designed to treat cancers and autoimmune diseases.

Other objects, features and advantages of the present invention will become apparent to those skilled in the art from the following detailed description. It is to be understood, however, that the detailed description and specific examples, while indicating some embodiments of the present invention, are given by way of illustration and not limitation. Many changes and modifications within the scope of the present invention may be made without departing from the spirit thereof, and the invention includes all such modifications.

### BRIEF DESCRIPTION OF THE DRAWINGS

FIG. 1. A feeder-free, serum-free culture method to support HSC-derived human T cell differentiation culture (denoted as Diff-T method), (A) Schematic showing Diff-T culture system to differentiate human CD34⁺ HSCs into T cells. Note: γδ T cells are generated prior to αβ T cells. (B) Table showing T cell subsets generated from CD34⁺ HSCs using the Diff-T culture method.

FIG. 2. Human T cell differentiation kinetics using the Diff-T culture method. (A) FACS analysis of T cell differentiation over 8 weeks. (B) Quantification of (A) (n=3; 3 CD34⁺ HSC donors).

FIG. 3. Successful generation of human γδ T (Diff-γδT) cells and their subsets using the Diff-T culture method. Week 6 Diff-T cell culture was stimulated with 1 μg/ml anti-CD3/CD28 for 3 days in the presence of irradiated healthy donor PBMCs as antigen presenting cells in Diff-T M3 medium supplemented with 10 ng/ml IL-7 and 10 ng/ml IL-15. 3 days after stimulation, Diff-T cell culture was washed and cultured without anti-CD3/CD28 for additional 11 days followed by flow cytometry analysis. (A) FACS detection of Diff-γδT cells (gated as CD3+TCRγδ+ cells). (B) FACS quantification of Diff-γδT cells over time. (C) FACS detection of γδ T cell subsets. Based on delta chain usage, Diff-γδT cells were classified as Vδ1 T cells, Vδ2 T cells, and others (cells using other γδTCRs). Data are presented as the mean ±SEM. ****P<0.0001, by Student's t test (B).

FIG. 4. Successful generation of functional human Vδ1 T (Diff-Vδ1 T) cells using the Diff-T culture method. Week 6 Diff-T cell culture was stimulated with 1 μg/ml anti-CD3/CD28 for 3 days in the presence of irradiated healthy donor PBMCs as antigen presenting cells in Diff-T M3 medium supplemented with 10 ng/ml IL-7 and 10 ng/ml IL-15. 3 days after stimulation, Diff-T cell culture was washed and cultured without anti-CD3/CD28 for additional 11 days followed by flow cytometry analysis. (A) FACS detection of Diff-Vδ1 T cells (gated as CD3+Vδ1+ cells). (B) Fold expansion of Diff-Vδ1 T cells. (C) FACS analysis showing the intracellular production of IFN-γ, granzyme B, and perforin in Diff-Vδ1 T cells. Data are presented as the mean ±SEM. ****P<0.0001, by Student's t test (B).

FIG. 5. Successful generation of functional human Vδ2 T (Diff-Vδ2 T) cells using the Diff-T culture method. Week 6 Diff-T cell culture was stimulated with 1 μg/ml anti-CD3/CD28 for 3 days in the presence of irradiated healthy donor PBMCs as antigen presenting cells in Diff-T M3 medium supplemented with 10 ng/ml IL-7 and 10 ng/ml IL-15. 3 days after stimulation, Diff-T cell culture was washed and cultured without anti-CD3/CD28 for additional 11 days followed by flow cytometry analysis. (A) FACS detection of Diff-Vδ2 T cells (gated as Vγ9+Vδ2+ cells). (B) Fold expansion of Diff-Vδ2 T cells. (C) FACS analysis showing the intracellular production of IFN-γ, granzyme B, and perforin in Diff-Vδ2 T cells. Data are presented as the mean +SEM. ****P<0.0001, by Student's t test (B).

FIG. 6. Successful generation of functional human conventional CD4 helper T (Diff-CD4 Tc) and CD8 cytotoxic T (Diff-CD8 Tc) cells using the Diff-T culture method. (A) Schematics showing the experimental design used to generate HSC derived conventional Diff-T cells that are either CD4 SP or CD8 SP. Note that a CD4 SP induction step is added between Stage 2 (T Cell Differentiation) and Stage 3 (T Cell Expansion) to dictate the CD4 SP vs. CD8 SP Tc cell product. (B) FACS monitoring of the transition of week 7 Diff-T cells from CD4/CD8 DP to CD4 SP following CD4 induction, or their retention as CD4/CD8 DP in the absence of CD4 induction by week 8 of T cell differentiation. The week 7 cell culture was treated with CD4 induction for 16 hours, followed by changed back to fresh media devoid of CD4 induction and further cultured for an additional 6 days. Note, the CD4 SP lineage commitment transcription factor ThPOK was detected in CD4 SP αβ T cells. (C-D) Week 8 Diff-T cell cultures (CD4 SP αβ T cells and CD4/CD8 DP αβ T cells) were stimulated with 1 μg/ml anti-CD3/CD28 for 3 days in the presence of irradiated healthy donor PBMCs as antigen presenting cells in Diff-T M3 medium supplemented with 10 ng/ml IL-7 and 10 ng/ml IL-15. 3 days after stimulation, Diff-T cell culture was washed and cultured without anti-CD3/CD28 for additional 11 days followed by flow cytometry analysis. (C) FACS detection of Diff-CD4 and Diff-CD8 Tc cell products. (D) Fold expansion of Diff-CD4 and Diff-CD8 Tc cell products. Data are presented as the mean ±SEM. ****P<0.0001, by Student's t test (D).

FIG. 7. Generation and characterization of Th0/Th1-like and Th2-like Diff-CD4 Tc cells. (A) Schematics showing an embodiment of the invention used to generate HSC derived CD4 SP αβ T cells that are Th0/Th1-like or Th2-like. Note that a CD4 SP induction step is added between Stage 2 (T Cell Differentiation) and Stage 3 (T Cell Expansion) to dictate the CD4 SP αβ T cell lineage commitment, and an optional Th2 polarization step is added in Stage 3 to dictate the Th0/Th1-like or Th2-like function of the final Diff-CD4 Tc cell product. Stage 3 CD4 SP of T cells were stimulated with 1 μg/ml anti-CD3/CD28 antibodies for 3 days in Diff-T M3 medium supplemented with human IL-2 and irradiated healthy donor PBMCs as antigen presenting cells, then cells were washed and cultured for additional 11 days, resulting in a Th0/Th1-like Diff-CD4 Tc final cell product. For Th2 polarization, Stage 3 CD4 SP αβ T cells were stimulated under the same condition but with the addition of ImmunoCult Human Th2 Differentiation Supplement (StemCell Technologies) containing human IL-4 and anti-human IFN-γ, resulting in a Th2-like Diff-CD4 Tc final cell product. (B) FACS analyses of intracellular production of cytokines and effector molecules by the indicated cells. Various HSC derived Diff-Tc cell products were analyzed, including those induced to be CD8 SP, Th0/Th1-like CD4 SP, and Th2-like CD4 SP. Note Th2-polarized Diff-CD4 To cells exhibit a typical Th2 -like function, evidenced by their reduced production of Th1 cytokines like IFN-γ while enhanced production of Th2 cytokines line IL-4, in sharp contrast to the non-Th2 -polarized Diff-CD4 Tc cells that exhibit a typical Th0/Th1-like function (high production of IFN-γ while low production of IL-4). On the other hand, the Diff-CD8 Tc cells exhibit a typical cytotoxic function such as high production of IFN-γ, as well as cytotoxic molecules like perforin and granzyme B.

FIG. 8. Successful generation of human iNKT (Diff-iNKT) and MAIT (Diff-MAIT) cells using the Diff-T culture method. Week 6 Diff-T cell culture was stimulated with 1 μg/ml anti-CD3/CD28 for 3 days in the presence of irradiated healthy donor PBMCs as antigen presenting cells in Diff-T M3 medium supplemented with 10 ng/ml IL-7 and 10 ng/ml IL-15. 3 days after stimulation, Diff-T cell culture was washed and cultured without anti-CD3/CD28 for additional 11 days followed by RNA extraction and RT-PCR. (A) Schematic of experimental design. TCR-specific primer set was used to amplify iNKT TCRα (Vα24Jα18). (B) Schematic of experimental design. TCR-specific primer set was used to amplify MAIT TCRα (Vα7.2Jα33). (C) Representative DNA gel image showing the PCR products of human iNKT TCRα and MAIT TCRα.

FIG. 9. Successful generation of a full-spectrum of human γδ (Vδ1-Vδ8) T cells using the Diff-T culture method. Week 6 Diff-T cell culture was stimulated with 1 μg/ml anti-CD3/CD28 for 3 days in the presence of irradiated healthy donor PBMCs as antigen presenting cells in Diff-T M3 medium supplemented with 10 ng/ml IL-7 and 10 ng/ml IL-15. 3 days after stimulation, Diff-T cell culture was washed and cultured without anti-CD3/CD28 for additional 11 days followed by RNA extraction and RT-PCR. (A) Schematic of experimental design. TCR-specific primer sets were used to amplify γδ T cell TCRδ chains (Vδ1-Vδ8), (B) DNA gel image showing the PCR products of human γδ T cell TCRδ chains (Vδ1-Vδ2). (C) DNA gel image showing the PCR products of human γδ T cell TCRδ chains (Vδ3-Vδ5). (D) DNA gel image showing the PCR products of human γδ T cell TCRδ chains (Vδ6-Vδ8).

FIG. 10. Successful generation of CAR/IL15 gene engineered Diff-γδT cells (^{CAR15}Diff-γδT) by combining the Diff-T culture method. CD34⁺ HSCs were engineered with a lentiviral vector (pMNDW backbone) encoding mesothelin targeting CAR (MCAR) and IL-15 gene then put into Diff-T culture. Week 4 Diff-T cell culture resulting from MCAR and IL-15 gene engineered human HSCs were stimulated with 1 μg/ml anti-CD3/CD28 for 3 days in the presence of irradiated healthy donor PBMCs as antigen presenting cells in Diff-T M3 medium supplemented with 10 ng/ml IL-7 and 10 ng/ml IL-15. 3 days after stimulation, Diff-T cell culture was washed and cultured without anti-CD3/CD28 for additional 11 days. Note, week 4 Diff-T culture resulted only γδ T cells without αβ T cells. (A) Schematic showing the generation of ^{CAR15}Diff-γST and ^{CAR15}Diff-Tc cells from CD34⁺ HSCs. Note, engineering was performed at stage 0 (before T differentiation of CD34⁺ HSCs). (B) FACS detection of MCAR and IL-15 gene engineered Diff-γδT (^{MCAR15}Diff-γδT) cells. (C) Yield of ^{MCAR15}Diff-γδT cells (n=3; n indicates different CD34⁺ HSC donors). The tumor attacking potency of MCAR15Diff-γδT cells were studied using the established in vitro tumor killing assay. OVCAR8, a human ovarian cancer cell line, was engineered with firefly luciferase (Fluc) and green fluorescence protein (EGFP) dual reporters and the resulting OVCAR8-FG cell line was then utilized in the study. Data was measured by luminescence assay at 24 hrs post co-culture (n=3). (D) Experimental design. (E) Tumor killing efficacy of ^{MCAR15}Diff-γδT cells against OVCAR8-FG tumor cells (n=3). Data are presented as the mean ±SEM. ns, not significant; *p<0.05, ****p<0.0001 by Student's t test (C) or one-way ANOVA (E).

FIG. 11. Generation of allogeneic CAR-IL15-γδT cells (^{AlloCAR15}Diff-γδT cells) by combining the Diff-T culture method with γδT TCR, CAR, and IL-15 gene engineering. CD34⁺ HSCs were engineered with a lentiviral vector (pMNDW backbone) encoding γδT TCR, BCMA targeting CAR (BCAR), and IL-15 gene then put into Diff-T culture. Week 4 Diff-T cell culture resulting from engineered human HSCs were stimulated with irradiated K562-based artificial APC (aAPC) expressing BCMA for 14 days. (A) Diff-T culture method combined with γδT TCR, CAR, and IL-15 gene engineering to generate ^{AlloCAR15}Diff-γδT cells from CD34⁺ HSCs. Note, engineering was performed at stage 0 (before T differentiation of CD34⁺ HSCs). (B) Yield of γδT TCR, BCAR, and IL-15 gene engineered ^{Allo}Diff-γδT (^{AlloBCAR15}Diff-γδT) cells (n=3; n indicates different CD34⁺ HSC donors). (C) FACS detection of ^{AlloBCAR15}Diff-γδT cells. The tumor attacking potency of ^{AlloBCAR15}Diff-γδT cells were studied using the established in vitro tumor killing assay. MM1.S, a human multiple myeloma cell line, was engineered with firefly luciferase (Fluc) and green fluorescence protein (EGFP) dual reporters and the resulting MM.1S-FG cell line was then utilized in the study. Data was measured by luminescence assay at 24 hrs post co-culture with or without the presence of 10 μM ZOL (n=3). (D) Experimental design. (E) Tumor killing efficacy of ^{AlloBCAR15}Diff-γδT cells against MMIS-FG tumor cells (n=3). Data are presented as the mean ±SEM. ns, not significant; *p<0.05, ****p<0.0001 by one-way ANOVA (E).

FIG. 12. Generation of allogeneic CAR-IL15-iNKT cells (^{AlloCAR15}Diff-iNKT cells) by combining the Diff-T culture method with iNKT TCR, CAR, and IL-15 gene engineering. CD34⁺ HSCs were engineered with a lentiviral vector (pMNDW backbone) encoding iNKT TCR, BCAR, and IL-15 gene then put into Diff-T culture. Week 4 Diff-T cell culture resulting from engineered human HSCs were stimulated with irradiated aAPC expressing BCMA for 14 days. (A) Diff-T culture method combined with iNKT TCR, CAR, and IL-15 gene engineering to generate ^{AlloCAR15}Diff-iNKT cells from CD34⁺ HSCs. Note, engineering was performed at stage 0 (before T differentiation of CD34⁺ HSCs). (B) Yield of iNKT TCR, BCAR, and IL-15 gene engineered ^{Allo}Diff-iNKT (^{AlloBCAR15}Diff-iNKT) cells (n=3; n indicates different CD34⁺ HSC donors). (C) FACS detection of ^{AlloBCAR15}Diff-iNKT cells. The tumor attacking potency of ^{AlloBCAR15}Diff-iNKT cells were studied using the established in vitro tumor killing assay. MM1.S, a human multiple myeloma cell line, was engineered with CD1d, firefly luciferase (Fluc) and green fluorescence protein (EGFP) dual reporters, and the resulting MM.1S-CD1d-FG cell line was then utilized in the study. Data was measured by luminescence assay at 24 hrs post co-culture with or without the presence of 100 ng/mL αGC (n=3). (D) Experimental design. (E) Tumor killing efficacy of ^{AlloBCAR15}Diff-iNKT cells against MMIS-CD1d-FG tumor cells (n=3). Data are presented as the mean ±SEM. ns, not significant; *p<0.05, ****p<0.0001 by one-way ANOVA (E).

FIG. 13. Generation of allogeneic CAR-IL15-MAIT cells (^{AlloCAR15}Diff-MAIT cells) by combining the Diff-T culture method with MAIT TCR, CAR, and IL-15 gene engineering. CD34⁺ HSCs were engineered with MAIT TCR, BCAR, and IL-15 gene then put into Diff-T culture. Week 4 Diff-T cell culture resulting from engineered human HSCs were stimulated with irradiated aAPC expressing BCMA for 14 days. (A) Diff-T culture method combined with MAIT TCR, CAR, and IL-15 gene engineering to generate ^{AlloCAR15}Diff-MAIT cells from CD34⁺ HSCs. Note, engineering was performed at stage 0 (before T differentiation of CD34⁺ HSCs). (B) Yield of MAIT TCR, BCAR, and IL-15 gene engineered ^{Allo}Diff-MAIT (^{AlloBCAR15}Diff-MAIT) cells (n=3; n indicates different CD34⁺ HSC donors). (C) FACS detection of ^{AlloBCAR15}Diff-MAIT cells. The tumor attacking potency of ^{AlloBCAR15}Diff-MAIT cells were studied using the established in vitro tumor killing assay. MM.1S-FG cell line was utilized in the study. Data was measured by luminescence assay at 24 hrs post co-culture with or without the presence of 840 nM 5-OP-RU (n=3). (D) Experimental design. (E) Tumor killing efficacy of ^{AlloBCAR15}Diff-MAIT cells against MM1S-FG tumor cells (n=3). Data are presented as the mean ±SEM. ns, not significant; *p<0.05, ****p<0.0001 by one-way ANOVA (E).

### DETAILED DESCRIPTION OF THE INVENTION

In the description of embodiments, reference may be made to the accompanying figures which form a part hereof, and in which is shown by way of illustration a specific embodiment in which the invention may be practiced. It is to be understood that other embodiments may be utilized, and structural changes may be made without departing from the scope of the present invention. Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. Many of the aspects of the techniques and procedures described or referenced herein are well understood and commonly employed by those skilled in the art. The following text discusses various embodiments of the invention.

Stem cells, including hematopoietic stem cells (HSCs) and pluripotent stem cells (PSCs), can be genetically engineered and differentiated into different types of immune cells, providing a substantial resource for developing off-the-shelf cell therapies. See, e.g., Li et al., Cell Stem Cell. 2023;30(5):592-610 and Li et al., Trends Cancer. 2021;7(12):1059-1073 describe the current stages of stem cell-based cell therapies, including HSC-derived and PSC-derived conventional αβ T (Tc) cells, nature killer (NK) cells, invariant natural killer T (iNKT) cells, gamma delta T (γδT) cells, and mucosal-associated invariant T (MAIT) cells. “HSCs” refer to human CD34+ hematopoietic stem and progenitor cells (HSPCs or HSCs; both terms are alternatively used in this invention document), that can be directly isolated from cord blood or G-CSF-mobilized peripheral blood (CB HSCs or PBSCs), or derived from embryonic or induced pluripotent stem cells (ES-HSCs or iPS-HSCs).

As discussed below, embodiments of the invention include unique medias (termed herein “Diff-T media”) and methods of using such medias (termed herein “Diff-T methods”), to differentiate human T cells (termed herein “Diff-T cells”) from hematopoietic stem cell progenitors. In typical embodiments, the Diff-T media are serum-free, and the Diff-T culture methods are feeder-free (i.e., do not comprise feeder cells). As compared to conventional methods of generating HSC-T cells using a feeder-dependent culture (e.g., ATO culture), embodiments of the invention disclosed herein offer an ex vivo differentiation method that does not require feeder cells. These new methods and medias greatly improve processes for the scale-up production and GMP-compatible manufacturing of therapeutic cells for human applications. Embodiments of the methods for making various T cell phenotypes from HSC progenitors can includes the ex vivo differentiation of Diff-T cells without feeder cells; further the ex vivo expansion of these differentiated Diff-T cells.

Embodiments of the invention include unique medias (“Diff-T media”) and methods (“Diff-T methods”), that can be used to differentiate human T cells from hematopoietic stem cells. In typical embodiments of the invention, the Diff-T media are serum-free, and the Diff-T culture methods are feeder-free. Hematopoietic stem cells (HSCs) such as human CD34⁺ hematopoietic stem and progenitor cells useful in embodiments of the invention can be isolated from cord-blood, bone marrow, or G-CSF mobilized peripheral blood, or can be differentiated from pluripotent stem cells (e.g., embryonic stem cells, induced pluripotent stem cells).

The human T cells made by embodiments of the invention encompass all major T cell subtypes, including gamma delta T cells (e.g., delta 1 and delta 2 T cells), conventional alpha beta T cells (e.g., CD4+ helper T cells, CD8+ cytotoxic T cells), and unconventional alpha beta T cells (e.g., invariant natural killer T cells, mucosal associate-invariant T cells). In addition, in certain embodiments of the invention, gene engineering (gene-overexpression or gene-editing) can be incorporated in the Diff-T methods at various culture stages, including the HSC, T cell progenitor, or mature T cell stages, to generate gene-engineered human T cells. The resulting human T cells, gene engineered or non-engineered, can be used for T cell biology study, drug discovery, or T cell-based immunotherapy.

The invention disclosed herein has a number of embodiments. Embodiments of the invention include, for example, cell culture medias. For example, one embodiment of the invention is a mammalian cell culture media comprising IL-3, IL-6, IL-7, Flt3 ligand, SCF, TPO, SDF-1α, StemRegenin 1, UM171, and UM729; wherein hematopoietic stem cells disposed in the cell culture media grow and differentiate; and the cell culture media does not comprise serum and/or feeder cells. In certain embodiments of the invention, the cell culture media further comprises at least one of: canonical Notch ligand 1 (DLL-1), delta like canonical Notch ligand 4 (DLL-4), vascular cell adhesion molecule 1 (VCAM-1), vascular cell adhesion molecule 5 (VCAM-5), retronectin, serum albumin, recombinant human insulin, human transferrin, human LDL, L-ascorbic acid, valproic acid, 2-mercaptoethanol. In certain embodiments of the invention, the cell culture media comprises a buffered medium comprising canonical Notch ligand 1 (DLL-1) or delta like canonical Notch ligand 4 (DLL-4), vascular cell adhesion molecule 1 (VCAM-1) or vascular cell adhesion molecule 5 (VCAM-5), retronectin, serum albumin, recombinant human insulin, human transferrin, human LDL, L-ascorbic acid, valproic acid, 2-mercaptoethanol, IL-3, IL-6, IL-7, Flt3 ligand, SCF, TPO, SDF-1 α, StemRegenin 1, UM171, and UM729. In some embodiments of the invention, a cell culture media disclosed herein comprises human hematopoietic stem cells. In certain embodiments of the invention, a cell culture media disclosed herein comprises human hematopoietic stem cells differentiated into at least one cellular phenotype selected from αβ CD8+ T cells; αβ CD4+ T cells; Type 1 invariant natural killer T cells; Type 2 natural killer T cells; MAIT cells; and γδ T cells. In certain of these embodiments, the human hematopoietic stem cells do not comprise a genetically modified genome. In other embodiments, the human hematopoietic stem cells do comprise a genetically modified genome including an exogenous nucleic acid sequence.

Another embodiment of the invention is a mammalian cell culture media comprising mammalian cell culture media comprising IL-7, IL-15, TGF-β, Flt3 ligand, SCF, SDF-1α, and MCP-4; wherein hematopoietic stem cells disposed in the cell culture media grow and differentiate; and the cell culture media does not comprise serum and/or feeder cells. In typical embodiments of the invention, the media comprises canonical Notch ligand 1 (DLL-1) or delta like canonical Notch ligand 4 (DLL-4), vascular cell adhesion molecule 1 (VCAM-1) or vascular cell adhesion molecule 5 (VCAM-5), and retronectin. In certain embodiments of the invention, the cell culture media further comprises at least one of: serum albumin, recombinant human insulin, human transferrin, human LDL, L-ascorbic acid, valproic acid, and 2-mercaptoethanol. In some embodiments of this invention, this cell culture media further comprises human hematopoietic stem cells; and/or human hematopoietic stem cells differentiated into at least one cellular phenotype selected from αβ CD8+ T cells; αβ CD4+ T cells; Type 1 invariant natural killer T cells; Type 2 natural killer T cells; MAIT cells; and γδ T cells.

Embodiments of the invention further comprise methods of culturing and/or differentiating hematopoietic stem cells comprising disposing hematopoietic stem cells into a first cell culture media comprising IL-3, IL-6, IL-7, Flt3 ligand, SCF, TPO, SDF-1α, StemRegenin 1, UM171, and UM729 such that the hematopoietic stem cells differentiate. Typically, these methods further comprise disposing the hematopoietic stem cells into a second cell culture media comprising: IL-7, IL-15, TGF-β, Flt3 ligand, SCF, SDF-1α, and MCP-4 such that the hematopoietic stem cells differentiate. In certain embodiments of the invention, the method is selected to differentiate hematopoietic stem cells into at least one cellular phenotype selected from αβ CD8+ T cells; αβ CD4+ T cells; Type 1 invariant natural killer T cells; Type 2 natural killer T cells; MAIT cells; and γδ T cells. In some embodiments of the invention, the method is selected to differentiate hematopoietic stem cells into unconventional T cells targeting MHC class Ib molecules such as HLA-E.

In certain embodiments of the invention, a differentiated T cell made by the methods disclosed herein does not comprise an exogenous nucleic acid molecule. In other embodiments, a nucleic acid is introduced into cells such as one encoding one or more T cell receptor amino acids. This can be done by transfecting or infecting the cell with a recombinant vector, which may or may not be a viral vector as discussed herein. The exogenous nucleic acid may incorporate into the cell's genome in some embodiments. In additional embodiments, a nucleic acid further comprises a nucleic acid sequence encoding a suicide gene product. In some embodiments, a nucleic acid molecule that is introduced into a selected CD34⁺ cell encodes a TCR polypeptide, and a suicide gene product. In other embodiments, a method also involves introducing into the selected CD34⁺ cells a nucleic acid encoding a suicide gene product, in which case a different nucleic acid molecule encodes the suicide gene product than a nucleic acid encoding at least one of the TCR polypeptide.

Certain embodiments of the invention are designed to selectively drive hematopoietic stem cells into one or more specific T cell phenotypes, for example, by culturing the cells for a selected period of time in a media disclosed herein and/or by engineering the cells (e.g., by transducing the cells with exogenous polynucleotides encoding one or more T cell receptors) in a manner that drives the hematopoietic stem cells towards a specific T cell phenotype. In certain embodiments of the invention, the cells are disposed in the first cell culture media for at least 2-10 weeks; and/or the cells are disposed in the second cell culture media for at least 2-10 weeks. In some embodiments of the invention, the cells are disposed in the first cell culture media for not more than 2-10 weeks; and/or the cells are disposed in the second cell culture media for not more than 2-10 weeks. In some embodiments of the invention, the method is selected to differentiate the hematopoietic stem cells into αβ CD8+ T cells, for example by transducing hematopoietic stem cells with polynucleotides encoding one or more conventional α and/or β T cell receptor gene sequences. In some embodiments of the invention, the method is selected to differentiate the hematopoietic stem cells into Type 1 invariant natural killer T cells, for example by transducing hematopoietic stem cells with polynucleotides encoding one or more invariant natural killer T cell receptor gene sequences. In some embodiments of the invention, the method is selected to differentiate the hematopoietic stem cells into MAIT cells, for example by transducing hematopoietic stem cells with polynucleotides encoding one or more semi-invariant T cell receptor alpha (TCRα) chain gene sequences. In some embodiments of the invention, the method is selected to differentiate the hematopoietic stem cells into γδ T cells, for example by transducing hematopoietic stem cells with polynucleotides encoding one or more γ and/or δ T cell receptor gene sequences.

In some embodiments the differentiated T cells do not express the HLA-I and/or HLA-II molecules on the cell surface, which may be achieved by disrupting the expression of genes encoding beta-2-microglobulin (B2M), transactivator (CIITA), or HLA-I and HLA-II molecules. In certain embodiments, methods involve eliminating surface expression of one or more HLA-I/II molecules in the isolated human CD34⁺ cells. In particular embodiments, eliminating expression may be accomplished through gene editing of the cell's genomic DNA. Some methods include introducing CRISPR and one or more guide RNAs (gRNAs) corresponding to B2M or CIITA into the cells. In particular embodiments, CRISPR or the one or more gRNAs are transfected into the cell by electroporation or lipid-mediated transfection. Consequently, methods may involve introducing CRISPR and one or more gRNAs into a cell by transfecting the cell with nucleic acid(s) encoding CRISPR and the one or more gRNAs. A different gene editing technology may be employed in some embodiments.

Human T cells that can be derived from HSCs using the medias and methods disclosed herein encompass all major T cell subtypes, including gamma delta T cells (e.g., delta 1 and delta 2 T cells), conventional alpha beta T cells (e.g., CD4+ helper T cells, CD8+ cytotoxic T cells), and unconventional alpha beta T cells (e.g., natural killer T cells, mucosal associate-invariant T cells). In some embodiments of the invention, the human hematopoietic stem cells do not comprise a genetically modified genome. In other embodiments of the invention, the human hematopoietic stem cells do comprise a genetically modified genome (e.g., a perturbation such as a deletion in the genome of these cells, and/or the inclusion of an exogenous nucleic acid sequence).

In certain embodiments of the invention, T cells are generated through gene-engineering (e.g., the addition of an exogenous TCR) of stem and progenitor cells (e.g., CD34+ HSCs, ESCs, iPSCs), followed by differentiation into engineered transgenic Diff-T cells. HSCs can be gene engineered via vector-dependent or vector-independent gene delivery methods, or via other gene editing methods known in the art (e.g., CRISPR, TALEN, Zinc finger). In certain embodiments of the invention, HSCs are engineered to express targeting molecules to enhance their disease-targeting capacity. Such targeting molecules can include Chimeric Antigen Receptors (CARs), natural or synthetic receptors/ligands, or others. The resulting CAR-engineered T cells can then be utilized for disease-targeting cellular therapy. In some embodiments of the invention, HSC derived Diff-T cells and derivatives are engineered to overexpress genes encoding T cell stimulatory factors, or to disrupt genes encoding T cell inhibitory factors, resulting in functionally enhanced HSC-T cells and derivatives. The resulting human T cells, gene engineered or non-engineered, can be used for T cell biology study, drug discovery, or T cell-based immunotherapy.

Certain methods of the invention involve culturing selected CD34⁺ cells in media (e.g., prior to introducing one or more nucleic acids into the cells). Culturing the cells can include incubating the selected CD34⁺ cells with media comprising one or more growth factors or similar agents. In some embodiments, one or more growth factors or similar agents comprise at least 2 or 3 or 4 of: IL-3, IL-6, IL-7, IL-15, TGF-β, Flt3 ligand, SCF, TPO, SDF-1α, MCP-4, StemRegenin 1, UM171, and UM729. Embodiments of the invention can include further agents such as c-kit ligand, flt-3 ligand, and/or human thrombopoietin (TPO). In further embodiments, the media includes other agents such as c-kit ligand and TPO. In some embodiments, the concentration of the one or more growth factors is between about 5 ng/ml to about 500 ng/ml with respect to either each growth factor or the total of any and all of these particular growth factors. The concentration of a single growth factor or the combination of growth factors in media can be about, at least about, or at most about 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 410, 420, 425, 430, 440, 441, 450, 460, 470, 475, 480, 490, 500 (or any range derivable) ng/ml or μg/ml or more.

In some embodiments of the invention, HSCs can be engineered to express a selected TCR gene followed by differentiation using Diff-T media and methods. The resulting cell products express monoclonal TCR with high purity and yield and are suitable for allogeneic adoptive transfer and therefore can be utilized as off-the-shelf cellular products. These cell products include ^{Allo}Diff-T cells (e.g., those engineered with TCR). In some embodiments, Diff-T cells can be engineered to express additional transgenes. In one embodiment, such transgenes encode disease targeting molecules such as T-cell receptors (TCRs), chimeric antigen receptors (CARs), and other native or synthetic receptor/ligands. In another embodiment, such transgenes can encode T cell regulatory proteins such as IL-2, IL-7, IL-15, IFN-γ, TNF-α, CD28, 4-1BB, OX40, ICOS, FOXP3, and others. Transgenes can be introduced into post-expansion of Diff-T cells or their progenitor cells (HSCs, newly differentiated Diff-T cells, in-expansion Diff-T cells) at various culture stages.

In some embodiments of the invention, Diff-T cells can be engineered to disrupt selected genes using gene editing tools (CRISPR, TALEN, Zinc-Finger, and others). In one embodiment, disrupted genes encode T cell immune checkpoint inhibitors (PD-1, CTLA-4, TIM-3, LAG-3, and others). Deficiency of these negative regulatory genes may enhance the disease fighting capacity of Diff-T cells, making them resistant to disease-induced anergy and tolerance.

In some embodiments of the invention, Diff-T cells or enhanced Diff-T cells can be further engineered to make them suitable for allogeneic adoptive transfer, thereby suitable for serving as off-the-shelf cellular products. In one embodiment, genes encoding MHC molecules or MHC expression/display regulatory molecules (MHC molecules, B2M, CIITA (Class II transcription activator control induction of MHC class II mRNA expression), and others). Lack of MHC molecule expression on Diff-T cells makes them resistant to allogeneic host T cell-mediated depletion. In another embodiment, MHC class-I deficient Diff-T cells will be further engineered to overexpress an HLA-E gene that will endow them resistant to host NK cell-mediated depletion.

In embodiments of the invention, Diff-T cells and derivatives can be used freshly or cryopreserved for further usage. Moreover, various intermediate cellular products generated during Diff-T cell culture can be paused for cryopreservation, stored and recovered for continued production.

In typical embodiments of the invention, cells are cultured in cell-free and/or serum free medium. In certain embodiments, the serum-free medium further comprises externally added ascorbic acid. In particular embodiments, methods involve adding ascorbic acid medium. In further embodiments, the serum-free medium further comprises 1, 2, 3, 4, 5, 6, 7, 8, 9, 10, 11, 12, 13, 14, 15, or all 16 (or a range derivable therein) of the following externally added components: FLT3 ligand (FLT3L), interleukin 7 (IL-7), stem cell factor (SCF), thrombopoietin (TPO), stem cell factor (SCF), IL-2, IL-4, IL-6, IL-15, IL-21, TNF-alpha, TGF-beta, interferon-gamma, interferon-lambda, TSLP, thymopentin, pleotrophin, or midkine. In additional embodiments, the serum-free medium comprises one or more vitamins. In some cases, the serum-free medium includes 1, 2, 3, 4, 5, 6, 7, 8, 9, 10, 11, or 12 of the following vitamins (or any range derivable therein): comprise biotin, DL alpha tocopherol acetate, DL alpha-tocopherol, vitamin A, choline chloride, calcium pantothenate, pantothenic acid, folic acid nicotinamide, pyridoxine, riboflavin, thiamine, inositol, vitamin B12, or a salt thereof. In certain embodiments, medium comprises or comprise at least biotin, DL alpha tocopherol acetate, DL alpha-tocopherol, vitamin A, or combinations or salts thereof. In additional embodiments, serum-free medium comprises one or more proteins. In some embodiments, serum-free medium comprises 1, 2, 3, 4, 5, 6 or more (or any range derivable therein) of the following proteins: albumin or bovine serum albumin (BSA), a fraction of BSA, catalase, insulin, transferrin, superoxide dismutase, or combinations thereof. In other embodiments, serum-free medium comprises 1, 2, 3, 4, 5, 7, 8, 9, 10, or 11 of the following compounds: corticosterone, D-Galactose, ethanolamine, glutathione, L-carnitine, linoleic acid, linolenic acid, progesterone, putrescine, sodium selenite, or triodo-I-thyronine, or combinations thereof. In further embodiments, serum-free medium comprises a B-27® supplement, xeno-free B-27® supplement, GS21™ supplement, or combinations thereof. In additional embodiments, serum-free medium comprises or further comprises amino acids, monosaccharides, and/or inorganic ions. In some aspects, serum-free medium comprises 1, 2, 3, 4, 5, 6, 7, 8, 9, 10, 11, 12, or 13 of the following amino acids: arginine, cysteine, isoleucine, leucine, lysine, methionine, glutamine, phenylalanine, threonine, tryptophan, histidine, tyrosine, or valine, or combinations thereof. In other aspects, serum-free medium comprises 1, 2, 3, 4, 5, or 6 of the following inorganic ions: sodium, potassium, calcium, magnesium, nitrogen, or phosphorus, or combinations or salts thereof. In additional aspects, serum-free medium comprises 1, 2, 3, 4, 5, 6 or 7 of the following elements: molybdenum, vanadium, iron, zinc, selenium, copper, or manganese, or combinations thereof.

Diff-T media and methods can be used to generate human T cells that encompass all major T cell subtypes, providing an opportunity to study T cell development and TCR discovery. The resulting T cells may also be utilized for different therapeutic purposes such as treating cancers and autoimmune diseases. T cell subtypes that can derived from HSCs in embodiments of the invention include the following:

### Conventional αβ CD8 T (CD8 T) Cells

CD8 T cells recognize protein peptide antigens presented by polymorphic major histocompatibility complex (MHC) Class I molecules. CD8 T cells are potent cytotoxic cells for killing target pathogenic cells. CD8 T cells are also termed cytotoxic T lymphocytes (CTLs).

### Conventional αβ CD4 T (CD4 T) Cells

CD4 T cells recognize protein peptide antigens presented by polymorphic MHC Class II molecules. CD4 T cells are helper T (TH) cells orchestrating the immune responses. They help CD8 T cells by supporting their expansion and differentiation into functional memory T cells. Based on their specialized functions, CD4 T cells can be classified into further subtypes: TH1, TH2, TH17, TFH, TH9, TREG, and more.

### CD4 T Helper Cells and Subsets

In human peripheral blood, CD4 T cells represent approximately two-thirds of the total T cell population, while CD8 T cells constitute the remaining third. All helper T (T_{H}) cells express CD4 co-receptors. Upon encountering antigens, naïve T_{H} cells become activated, undergo differentiation, and release cytokines to facilitate and modulate immune reactions. Depending on the cytokine milieu, naïve CD4 T_{H} cells have the capacity to polarize into various subsets, including T_{H1}, T_{H2}, TH₁₇, TH₉, T follicular helper cells (T_{FH}), and regulatory T (T_{REG}) cells as discussed below. See, e.g., Zhu X, Zhu J. CD4 T Helper Cell Subsets and Related Human Immunological Disorders. Int J Mol Sci. 2020;21(21):8011. Published 2020 Oct. 28. doi:10.3390/ijms21218011.

### T_{H1} Cells

T_{H1} cells are involved in cellular immunity. They are characterized by the transcription factor T-bet and signal transducer and activator of transcription (STAT) 4, and the production of IL-2, IFN-γ, and TNF-α.

### T_{H2} Cells

T_{H2} cells are mediators of humoral immunity. They develop into IL-4-, IL-5-, and IL-13-producing cells, and are characterized by expressing transcription factor GATA-3 and STAT6.

### TH₁₇ Cells

TH₁₇ cells produce IL-17 and regulate tissue inflammation in host defense and chronic diseases. They are characterized by expressing transcription factor RORY.

### T_{FH} Cells

T_{FH} cells are essential for germinal center formation, affinity maturation, and development of most high-affinity antibodies and memory B cells. They secrete IL-6, IL-10, IL-12, IL-21, and are characterized by expressing transcriptional factor Bcl6.

### T_{H9} Cells

T_{H9} cells produce IL-9 and play a role in defense against helminth infections, in allergic responses, in autoimmunity, and tumor suppression. They are characterized by expressing transcriptional factor PU.1.

### T_{REG} Cells

T_{REG} cells are known as suppressor T cells, are a subpopulation of T cells that modulate the immune system, maintain tolerance to self-antigens, and prevent autoimmune disease. They secrete anti-inflammatory cytokines IL-10, and TGF-β, and are characterized by expressing transcriptional factor Foxp3.

### CD4 T Helper Cell Polarization

Naïve CD4 T_{H} cells can be further polarized into different subsets by activating the cells. See, e.g., Knosp C A, Johnston J A. Regulation of CD4+ T-cell polarization by suppressor of cytokine signalling proteins. Immunology. 2012;135(2):101-111.doi:10.1111/j.1365-2567.2011.03520.x

### T_{H1} Cells

TCR and co-stimulatory molecule activation (e.g., anti-CD3/CD28 antibodies), supporting cytokine IL-2, with the addition of IL-12, IL-18, and anti-CD4 antibody (e.g., ImmunoCult Human Th1 Differentiation Supplement).

### T_{H2} Cells

TCR and co-stimulatory molecule activation (e.g., anti-CD3/CD28 antibodies), supporting cytokine IL-2, with the addition of with IL-4 and anti-IFN-γ antibody (e.g., ImmunoCult Human Th2 Differentiation Supplement).

### T_{H17} Cells

TCR and co-stimulatory molecule activation (e.g., anti-CD3/CD28 antibodies), supporting cytokine IL-2, with the addition of TGF-β, IL-β, IL-6, IL-23, anti-IFN-γ antibody, and anti-CD4 antibody.

### T_{REG} Cells

TCR and co-stimulatory molecule activation (e.g., anti-CD3/CD28 antibodies), supporting cytokine IL-2, with the addition of TGF-β and All-trans retinoic acid (e.g., ImmunoCult™ Human Treg Differentiation Supplement).

### Type 1 Invariant Natural Killer T (iNKT) Cells

iNKT cells recognize glycolipid antigens presented by a non-polymorphic non-classical MHC Class I-like molecule CD1d. Consequently, iNKT cells do not cause graft-versus-host disease (GvHD) when adoptively transferred into allogeneic recipients. iNKT TCR comprises an invariant alpha chain (Vα14-Jα18 in mouse; Vα24-Jα18 in human), and a limited selection of beta chains (predominantly Vβ8/Vβ7/Vβ2 in mouse; predominantly Vβ11 in human). Both mouse and human iNKT cells respond to a synthetic agonist glycolipid ligand, alpha-Galactosylceramide (αGC, or α-GC, or α-GalCer).

### Type 2 Natural Killer T (NKT) Cells

Type 2 NKT cells are also restricted to CD1d. Type 2 NKT cells have a more diverse TCR repertoire, and their antigens are less well defined.

### MAIT Cells

Human mucosal-associated invariant T (MAIT) cells are characterized by their expression of an invariant TCRα chain Vα7.2-Jα33/Jα20/Jα12 paired with a restricted TCRβ chain. MAIT cells recognize microbial peptides presented by the highly conserved MHC class I-like molecule MR1 and bridge the innate and acquired immune systems to mediate augmented immune responses. Upon activation, MAIT cells rapidly proliferate, produce a variety of cytokines and cytotoxic molecules, and trigger efficient antitumor immunity. Administration of a representative MAIT cell ligand 5-OP-RU effectively activates MAIT cells and enhances their antitumor capacity. See, e.g., Li Y R, Zhou K, Wilson M, et al. Mucosal-associated invariant T cells for cancer immunotherapy. Mol Ther. 2023, and Godfrey D. I., Le Nours J., Andrew D. M., Uldrich A. P., and Rossjohn J. 2018. Unconventional T cell targets for cancer immunotherapy. Immunity 48, Mar. 20, 2018.)

### γδ T Cells

The human γδ T subsets that have been most comprehensively studied are Vδ1+ and Vδ2+ T cells. γδ T cells that express the Vγ9 chains paired with the Vδ2 chains (Vγ9Vδ2 T cells) are the major γδ T cell population in human peripheral blood. Vγ9Vδ2 T cells react to cells with accumulated intracellular phosphoantigens (pAgs), intermediate metabolites produced by infected or transformed cells. These metabolites include isopentenyl pyrophosphate (IPP) formed by the mevalonate (MVA) pathway of tumor cells, and (E)-4-Hydroxy-3-methyl-but-2-enyl pyrophosphate (HMB-PP) produced by microbial isoprenoid biosynthesis. Overproduction of IPP in cancer cells as a result of dysregulated MVA pathway leads to activation of Vγ9Vδ2 T cells. Stimulation of Vγ9Vδ2 T cells can be achieved with bisphosphonates, a class of drugs that prevent or slow down bone loss, such as zoledronate (ZOL). Vδ1+ subset has been found to recognize CD1 proteins, although recognition of lipid antigens on CD1 molecules is typically associated with NKT cells. See, e.g., Lee D, Rosenthal C J, Penn N E, Dunn Z S, Zhou Y, Yang L. Human γδ T Cell Subsets and their Clinical Applications for Cancer Immunotherapy. Cancers (Basel). 2022 and Godfrey D. I., Le Nours J., Andrew D. M., Uldrich A. P., and Rossjohn J. 2018. Unconventional T cell targets for cancer immunotherapy. Immunity 48, Mar. 20, 2018.

### Other T Cells

See, e.g., Godfrey D. I., Le Nours J., Andrew D. M., Uldrich A. P., and Rossjohn J. 2018. Unconventional T cell targets for cancer immunotherapy. Immunity 48, Mar. 20, 2018.)

The cell products made by the medias and methods disclosed herein (Diff-T cells), display phenotypes/functionalities distinct from that of their native counterpart T cells as well as their counterpart T cells generated using other ex vivo culture methods (e.g., ATO culture method), making Diff-T cells unique cellular products.

Unique features of embodiments of the invention disclosed herein include the following.
1) Embodiments of the invention are Ex Vivo and Xenogeneic feeder-Free.
2) Embodiments of the invention can generate human CD8 single positive and CD4 single positive T cells. The CD4 SP T cells can be further polarized into T_{H2} T cells. Similarly, embodiments of the invention can generate γδ T (Vδ1 and Vδ2) cells, NKT cells, and MAIT cells. These T cells can express an endogenously rearranged TCR.
3) Embodiments of the invention generate human γδ T cells (e.g., Vδ1 and Vδ2 T cells), NKT cells, and MAIT cells. And these T cells express endogenously rearranged TCR!
4) Embodiments of the invention can be used for T cell development and TCR discovery study.
5) In embodiments of the invention, cells can live and differentiate in the culture up to 10 weeks, providing an opportunity for the cells to mature and differentiate better than occurs in conventional cultures.
6) Embodiments of the invention can support the synchronized differentiation of transgenic ^{Allo}Diff-T cells, thereby eliminating the presence of un-differentiated progenitor cells and other lineages of bystander immune cells.
7) Embodiments of the invention generate a high yield! About 10¹³ Diff-T cells (10,000-100,000 doses) can be generated from CD34+ HSCs of a healthy donor.
8) Embodiments of the invention generate can engineer cells with transgenic TCRs, CARs, and other molecules.

A number of illustrative working embodiments of the invention are described in the Examples below, which show the successful generation of Diff-αβT (CD8 and CD4), Diff-γδT (Vδ1 and Vδ2), Diff-NKT, and Diff-MAIT cells. Engineering of Diff-γδT cells to express a Mesothelin CAR (MCAR) and together with Interleukin-15 (IL-15) (MCAR15 Diff-γδT cell product) were also proved successful. Additionally, cells engineered with γδT TCR, BCMA CAR (BCAR), and IL-15 (Allo15BCAR Diff-γδT) were generated using Diff-T media and methods. Pilot CMC, pharmacology, and efficacy studies were performed analyzing these cell products.

### Illustrative Media and Methods

In certain embodiments of the invention, stage 1 Diff-T Medium (Diff-T M1) can be used for culturing CD34+ HSCs. Typically, this stage 1 Diff -T Medium media comprises a base medium containing serum albumin, recombinant human insulin, human transferrin, human LDL, L-ascorbic acid, valproic acid, 2-mercaptoethanol, IL-3, IL-6, IL-7, Flt3 ligand, SCF, TPO, SDF-1α, StemRegenin 1, UM171, UM729, and the like.

In certain embodiments of the invention, stage 2 Diff-T Medium (Diff-T M1) can also be used for culturing CD34+ HSCs. Typically stage 2 Diff-T Medium (Diff-T M2) comprises a base medium containing serum albumin, recombinant human insulin, human transferrin, human LDL, L-ascorbic acid, 2-mercaptoethanol, IL-7, IL-15, TGF-β, Flt3 ligand, SCF, SDF-1α, MCP-4, and the like.

In certain embodiments of the invention, T cell Expansion Medium can also be used for culturing cells. Typically, a T cell Expansion Medium used for Diff-T cell expansion can comprise a T cell culture media supplemented with supporting cytokines such as IL-2, IL-15, IL-7, IL-21, IL-12, and IL-18.

Illustrative embodiments of medias and methods of the invention are discussed in the following sections.

### Stage 1: Diff-T Cell Differentiation

Methodological embodiments of the invention include a first step or stage. Typically, in this stage, fresh or frozen/thawed CD34⁺ HSCs are cultured and differentiated in Diff-T M1 and Diff-T M2 media over a period of 4-12 weeks without feeders. Non-tissue culture-treated plates can be coated with a Diff-T Culture Coating Material (DLL-1/4, VCAM-1/5, retronectin, and others). CD34+ HSCs are suspended in a Diff-T M1 Medium and seeded into the coated wells of a plate and cultured for 1-3 weeks. Diff-T M1 Medium can be refreshed every 3-4 days. Cells can then be collected and suspended in a Diff-T M2 Medium and seeded into the coated wells of a plate and cultured for another 1-7 weeks. Typically, Diff-T M2 Medium is refreshed every 3-4 days.

In illustrative embodiments, a Stage 1 Diff-T Medium (Diff-T M1) used for culturing CD34+ HSCs was made of base medium (e.g., Iscove's Modified Dulbecco's Medium, RPM1 1640, SFEMII, αMEM, and others) containing a serum-free supplement (e.g., recombinant human albumin, BIT 9500 serum substitute, B-27 supplement, and others), Insulin-transferrin-selenium, 2-mercaptoethanol, Vitamin C (e.g., L-Ascorbic acid, L-Ascorbic acid 2-phosphate sesquimagnesium salt hydrate, and its other forms), human low density lipoproteins, IL-7, SCF, TPO, IL-3, IL-6, Flt3 ligand, SDF-1α, HSC self-renewal agonists (e.g., UM171, UM729, and others), aryl hydrocarbon receptor inhibitors (e.g., PD98059, BAY-218, StemRegenin 1, CH-223191, and others), p38 MAPK inhibitors (e.g., Adezmapimod, Doramapimod, Losmapimod, SB202190, and others).

### Stage 2: Diff-T Cell Expansion

Methodological embodiments of the invention include additional differentiation and/or expansion steps or stages. In certain embodiments in these steps, differentiated Diff-T cells are stimulated with TCR cognate antigens (proteins, peptides, lipids, phosphor-antigens, small molecules, and others) or non-specific TCR stimulatory reagents (anti-CD3/anti-CD28 antibodies or antibody-coated beads, Concanavalin A, PMA/Ionomycin, and artificial APCs), and expanded for up to 1 month in T cell Expansion Medium supplemented with supporting cytokines and additives.

In illustrative embodiments, a Stage 2 Diff-T Medium (Diff-T M2) was made of base medium (e.g., Iscoves Modified Dulbecco's Medium, RPM1 1640, SFEMII, αMEM, and others) containing a serum-free supplement (e.g., recombinant human albumin, BIT 9500 serum substitute, B-27 supplement, and others), Insulin-transferrin-selenium, 2-mercaptoethanol, Vitamin C (e.g., L-Ascorbic acid, L-Ascorbic acid 2-phosphate sesquimagnesium salt hydrate, and its other forms), human low density lipoproteins, IL-7, SCF, Flt3 ligand, IL-2, IL-15, IL-21, TGF-β, SDF-1α, HSC self-renewal agonists (e.g., UM171, UM729, and others), aryl hydrocarbon receptor inhibitors (e.g., PD98059, BAY-218, StemRegenin 1, CH-223191, and others), p38 MAPK inhibitors (e.g., Adezmapimod, Doramapimod, Losmapimod, SB202190, and others), and others.

In illustrative embodiments, a Stage 3 T cell Expansion Medium (Diff-T M3) used for Diff-T cell expansion was made of a base medium, including but not limited to, CTS OpTmizer, TexMACS, RPMI, DMEM, X-Vivo15, and supplemented with T cell supporting cytokines (e.g., IL-2, IL-4, IL-7, IL-12, IL-15, IL-18, IL-21, TNFα, TL-1A, SDF-1α, TGF-β, and others), small molecules and additives (e.g., Wnt activators or glycogen synthase kinase-3 (GSK-3) inhibitors such as Wnt3A, CHIR99021, AR-A014418, TWS119, LY2090314, 9-ING-41, lithium chloride (LiCl), BIO (6-bromoindirubin-3-oxime, 6-Bromoindirubin-3′-oxime, or tyrosine kinase inhibitors such as dasatinib, ibrutinib, acalabrutinib, and zanubrutinib, or caspase inhibitors such as Emricasan, Z-VAD-FMK, and Z-VKD-FMK). The cell culture approach can be serum-free and feeder-free.

Typically in Stage 1 and Stage 2: Diff-T cell differentiation steps, fresh or frozen/thawed CD34+ HSCs are cultured and differentiated in Diff-T M1 and Diff-T M2 medium over a period of 4-10 weeks without feeders. Non-tissue culture-treated plates are coated with a Diff-T Culture Coating (Diff-Tc) Material (DLL-1/4, VCAM-1/5, retronectin, and others). CD34+ HSCs are suspended in a Diff-T M1 Medium and seeded into the Diff-Tc coated wells of a plate, and cultured for about 2 weeks. Diff-T M1 Medium is refreshed every 3-4 days. Cells are then collected and suspended in a Diff-T M2 Medium and seeded into the Diff-Tc coated wells of a plate, and cultured for another 2-8 weeks. Diff-T M2 Medium is refreshed every 3-4 days.

Typically in Stage 3: Diff-T cell expansion steps, differentiated Diff-T cells are stimulated with TCR cognate antigens (proteins, peptides, lipids, phosphor-antigens, small molecules, and others) or non-specific TCR stimulatory reagents (anti-CD3/anti-CD28 antibodies or antibody-coated beads, Concanavalin A, PMA/Ionomycin, and artificial APCs), and expanded for up to 1 month in Diff-T M3 medium supplemented with supporting cytokines and additives.

Embodiments of the invention can include further steps such as a CD4 induction step. For example, at the end of the Stage 2 culture (Diff-T cell differentiation) step, when the developing Diff-T cells reach the CD4/CD8 double-positive (DP) stage, they can be further prompted to transition into CD4 SP Diff-T cells by culturing them on a Diff-Tc coated plate with CD4 inducer (CD4i) Medium. This specialized medium, comprising Diff-T M2 Medium supplemented with T cell activation molecules such as anti-CD3/CD28/CD2 antibodies, anti-CD3/CD28/CD2 beads, TCR stimulating antigens, phorbol 12-myristate 13-acetate (PMA) and ionomycin, as well as phytohaemagglutinin (PHA), is employed for a period of 10-48 hrs to transiently activate the cells. Following this activation phase, the CD4i Medium is replaced with fresh Diff-T M2 Medium, and the cells are cultured for an additional 2-7 days. The Diff-T M2 Medium is replenished every 3-4 days.

Embodiments of the invention can include further steps such as a T_{H} polarization step. For example, during stage 3 culture (Diff-T cell expansion), differentiated CD4 SP Diff-T cells can also be further polarized into different subsets of CD4 T cells, including T_{H1} (e.g., additionally add IL-12, IL-18, and anti-CD4 antibody), T_{H2} (e.g., additionally add IL-4 and anti-IFN-γ antibody), TH17 (e.g., additionally add TGF-β, IL-1β, IL-6, IL-23, anti-IFN-γ antibody, and anti-CD4 antibody), and T_{REG} (e.g., additionally add TGF-β and All-trans retinoic acid) cells.

In some embodiments of the invention, Diff-T cells can be further engineered to express additional transgenes. In one embodiment, such transgenes encode disease targeting molecules such as chimeric antigen receptors (CARs), T-cell receptors (TCRs), and other native or synthetic receptor/ligands. In another embodiment, such transgenes can encode T cell regulatory proteins such as IL-2, IL-4, IL-7, IL-10, IL-12, IL-15, IL-18, IFN-γ, TNF-α, TL1A, CD27, CD28, 4-1BB, OX40, ICOS, DAP10, and others. In another embodiment, such transgenes can encode transcription factors such as Bcl11b, Batf3, ThPOK, FOXP3, Runx3, and others. Transgenes can be introduced into post-expansion Diff-T cells or their progenitor cells (HSCs, newly differentiated Diff-T cells, in-expansion Diff-T cells) at various culture stages.

In some embodiments of the invention, Diff-T cells can be further engineered to disrupt selected genes using gene editing tools (CRISPR, TALEN, Zinc-Finger, and others). In one embodiment, disrupted genes encode T cell immune checkpoint inhibitors (PD-1, CTLA-4, TIM-3, LAG-3, CD161, and others). In another embodiment, disrupted genes encode T cell regulatory proteins (e.g., TET2, PI3Kδ/γ, DGK, DNMT3a, Suv39h1, and others). Deficiency of these negative regulatory genes may enhance the disease fighting capacity of Diff-T cells, making them resistance to disease-induced anergy and tolerance.

In some embodiments of the invention, Diff-T cells or enhanced Diff-T cells can be further engineered to make them suitable for allogeneic adoptive transfer, thereby suitable for serving as off-the-shelf cellular products. In one embodiment, genes encoding MHC molecules or MHC expression/display regulatory molecules [MHC molecules, B2M, CIITA (Class II transcription activator control induction of MHC class II mRNA expression), and others]. Lack of MHC molecule expression on Diff-T cells makes them resistant to allogeneic host T cell-mediated depletion. In another embodiment, MHC class-I deficient Diff-T cells will be further engineered to overexpress an HLA-C, HLA-E, HLA-G, and CD47 gene that will endow them resistant to host NK cell-mediated depletion.

In some embodiments of the invention, Diff-T cell products and derivatives can be used freshly or cryopreserved for further usage. In addition, various intermediate cellular products generated during HSC-to-Diff-T cell culture can be paused for cryopreservation, stored and recovered for continued production.

Further aspects and embodiments of the invention are disclosed in the Examples below.

### EXAMPLES

### Example 1: Ex Vivo Generation of Hematopoietic Stem Cell-Differentiated Human Γa T (Diff-ΓΔt) and AB T (DIFF-ABT) Cells; Without Gene Engineering

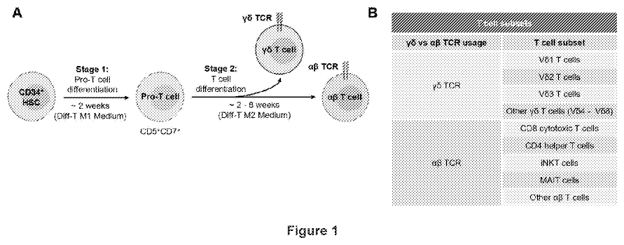

### Example 1A: A Feeder-Free, Serum-Free Culture Method (DIFF-T Method) to Support HSC-Derived Human T Cell Differentiation Culture (FIG. 1)

CD34⁺ HSCs isolated from G-CSF-mobilized peripheral blood (denoted as PBSCs), or cord blood (denoted as CB HSCs) were put into the feeder-free ex vivo Diff-T culture. The cells were cultured in Diff-T M1 media for about 2 weeks, then reseeded and cultured in Diff-T M2 media for another 2 to 10 weeks (FIG. 1A). Different subsets of T cells were generated in this culture, including CD8 single positive cytotoxic T cells, CD4 single positive helper T cells, γδ T cells (Vδ1, Vδ2, Vδ3, and others), iNKT cells, and MAIT cells (FIG. 1B).

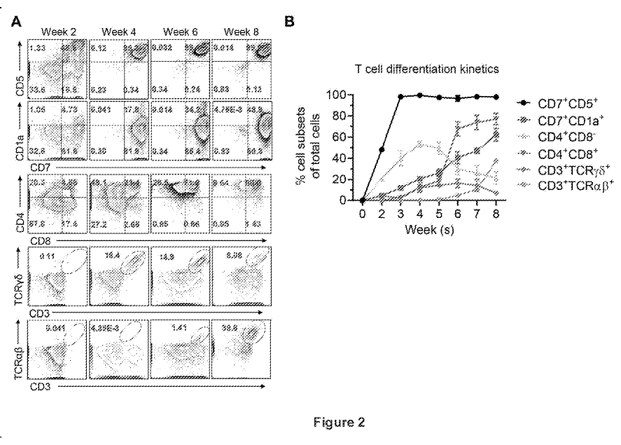

### Example 1B: Human T Cell Differentiation Kinetics Using the Diff-T Culture Method (FIG. 2)

The Diff-T culture method efficiently differentiated CD7+CD5+ progenitor T cells (pro-T cells) from CD34+ CB HSCs by week 2, and the differentiating cells subsequently gained CD1a expression, indicating the cells started to become pre-T cells (FIG. 2A). By week 3 to week 4, cells start to enter CD4+CD8+ DP stage, and most of them become DP by week 6. CD3+TCRγδ+ cells emerged at week 3, increased their numbers and reached plateau between week 4 to week 6. CD3+TCRαβ+ cells appeared at week 6, and eventually became the predominant population in the culture at week 8.

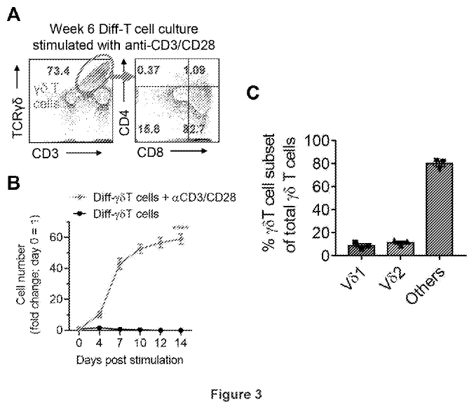

### Example 1C: Successful Generation of Human γδ T cells (Diff-γδT) and Their Subsets Using the Diff-T Culture Method (FIG. 3)

Week 6 Diff-T cell culture was stimulated with 1 μg/ml anti-CD3/CD28 for 3 days in the presence of irradiated healthy donor PBMCs as antigen presenting cells in Diff-T M3 medium supplemented with 10 ng/ml IL-7 and 10 ng/ml IL-15, 3 days after stimulation, Diff-T cell culture was washed and cultured without anti-CD3/CD28 for additional 11 days. Note, the detection of γδ T cells expressed CD4−CD8− double negative (DN) and CD4-CD8+ single positive (SP) populations (FIG. 3A) and expanded for about 60 folds (FIG. 3B). The total amount of TCR Vδ1 and TCR Vδ2 comprised about 20% of total γδ T cells, and other TCRγδ+ cells that expressed other Vδ chains had about 80% (FIG. 3C).

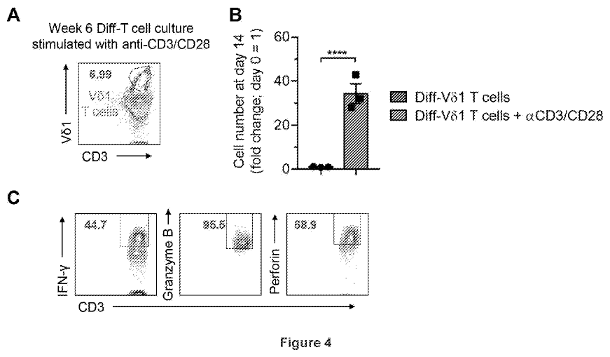

### Example 1D: Successful Generation of Functional Human Vδ1 T (Diff-Vδ1 T) Cells Using the Diff-T Culture Method (FIG. 4)

Week 6 Diff-T cell culture was stimulated with 1 μg/ml anti-CD3/CD28 for 3 days in the presence of irradiated healthy donor PBMCs as antigen presenting cells in Diff-T M3 medium supplemented with 10 ng/ml IL-7 and 10 ng/ml IL-15. 3 days after stimulation, Diff-T cell culture was washed and cultured without anti-CD3/CD28 for additional 11 days. Diff-Vδ1 T cells (FIG. 4A) expanded for about 35 folds (FIG. 4B) and expressed IFN-γ, granzyme B, and perforin (FIG. 4C).

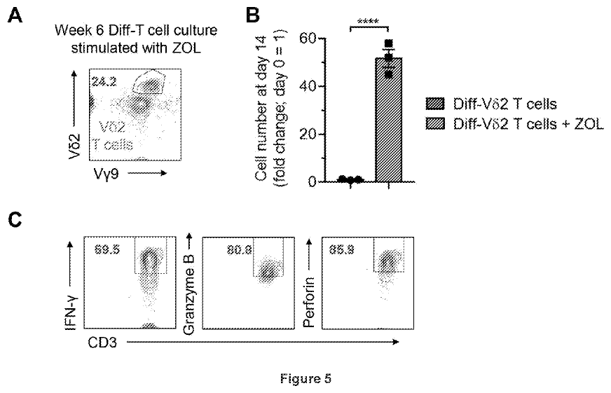

### Example 1E: Successful Generation of Functional Human Vδ2 T (Diff-Vδ2 T) Cells Using the Diff-T Culture Method (FIG. 5)

Week 6 Diff-T cell culture was stimulated with 5 μM zoledronate (ZOL) for 3 days in the presence of irradiated healthy donor PBMCs as antigen presenting cells in Diff-T M3 medium supplemented with 10 ng/ml IL-7 and 10 ng/ml IL-15. 3 days after stimulation, Diff-T cell culture was washed and cultured without ZOL for additional 11 days. Diff-Vδ2 (identified as Vγ9+Vδ2+) T cells (FIG. 5A) expanded for about 50 folds (FIG. 5B) and expressed IFN-γ, granzyme B, and perforin (FIG. 5C).

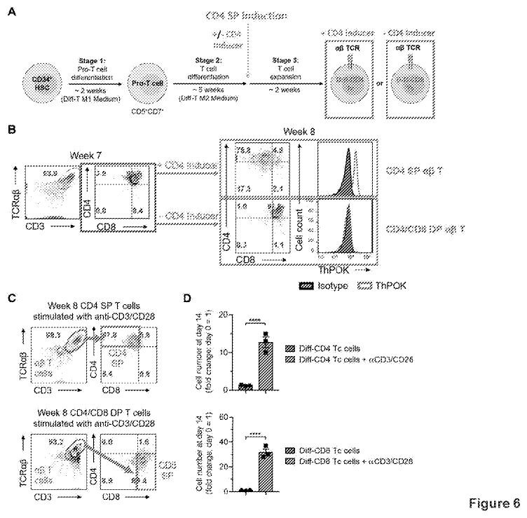

### Example 1F: Successful Generation of Functional Human Conventional CD4 Helper T (Diff-CD4 Tc) and CD8 Cytotoxic T (Diff-CD8 Tc) Cells Using the Diff-T Culture Method (FIG. 6)

FIG. 6(A) provides schematics showing one embodiment of the invention designed for generating HSC derived conventional Diff-Tc cells that are either CD4 SP or CD8 SP. Note that a CD4 SP induction step is added between Stage 2 (T Cell Differentiation) and Stage 3 (T Cell Expansion) to dictate the CD4 SP vs. CD8 SP of T cell product. FIG. 6(B) provides data from FACS monitoring of the transition of week 7 Diff-T cells from CD4/CD8 DP to CD4 SP following CD4 induction, or their retention as CD4/CDS DP in the absence of CD4 induction by week 8 of T cell differentiation. The week 7 cell culture was treated with CD4 induction for 16 hours, followed by changed back to fresh media devoid of CD4 induction and further cultured for an additional 6 days. Note, the CD4 SP lineage commitment transcription factor ThPOK was detected in CD4 SP αβ T cells. FIG. 6(C-D) show data from studies where week 8 Diff-T cell cultures (CD4 SP αβ T cells and CD4/CD8 DP αβ T cells) were stimulated with 1 μg/ml anti-CD3/CD28 for 3 days in the presence of irradiated healthy donor PBMCs as antigen presenting cells in Diff-T M3 medium supplemented with 10 ng/ml IL-7 and 10 ng/ml IL-15. 3 days after stimulation, Diff-T cell culture was washed and cultured without anti-CD3/CD28 for additional 11 days.

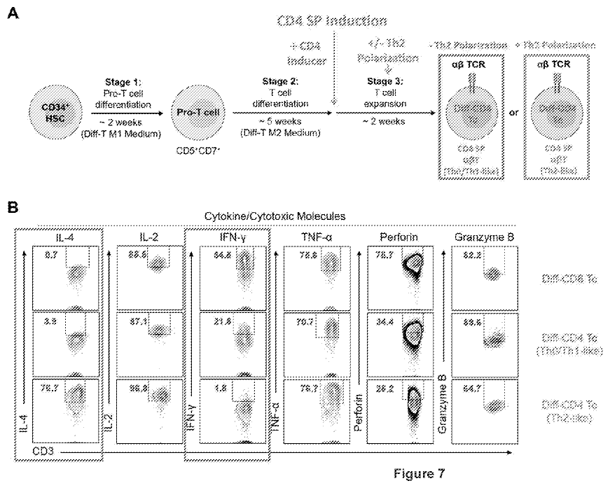

### Example 1G: Successful and Characterization of Th0/Th1-like and Th2-like Diff-CD4 Tc Cells (FIG. 7)

FIG. 7 provides schematics and data showing one embodiment for generating HSC derived Diff-CD4 Tc cells that are Th0/Th1-like or Th2-like. In this embodiment, a CD4 SP induction step is added between Stage 2 (T Cell Differentiation) and Stage 3 (T Cell Expansion) to dictate the CD4 SP αβ T cell lineage commitment, and an optional Th2 polarization step is added in Stage 3 to dictate the Th0/Th1-like or Th2-like function of the final Diff-CD4 Tc cell product. In one illustrative embodiment, Stage 3 CD4 SP αβ T cells were stimulated with 1 μg/ml anti-CD3/CD28 antibodies for 3 days in the presence of irradiated healthy donor PBMCs as antigen presenting cells in Diff-T M3 medium supplemented with human IL-2 and irradiated healthy donor PBMCs as antigen presenting cells, resulting in a Th0/Th1-like Diff-CD4 Tc final cell product. For Th2 polarization, Stage 3 CD4 SP αβ T cells were stimulated under the same condition but with the addition of ImmunoCult Human Th2 Differentiation Supplement (StemCell Technologies) containing human IL-4 and anti-human IFN-γ, resulting in a Th2-like Diff-CD4 Tc final cell product.

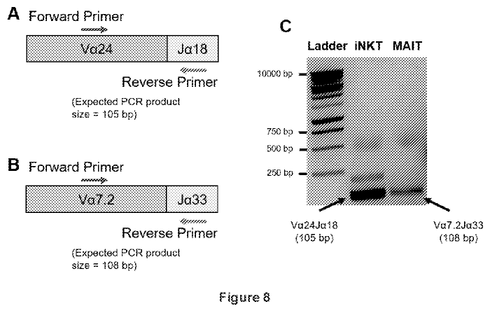

### Example 1H: Successful Generation of Human iNKT (Diff-iNKT) and MAIT (Diff-MAIT) Cells Using the Diff-T Culture Method (FIG. 8)

Week 6 Diff-T cell culture was stimulated with 1 μg/ml anti-CD3/CD28 for 3 days in the presence of irradiated healthy donor PBMCs as antigen presenting cells in Diff-T M3 medium supplemented with 10 ng/ml IL-7 and 10 ng/ml IL-15. 3 days after stimulation, Diff-T cell culture was washed and cultured without anti-CD3/CD28 for additional 11 days. RNA extraction and RT-PCR were then performed to detect the successfully rearranged signature invariant TCRα chain Vα24Jα18 utilized by invariant natural killer T (iNKT) cells and Vα7.2Jα33 utilized by mucosal-associated invariant T (MAIT) cells.

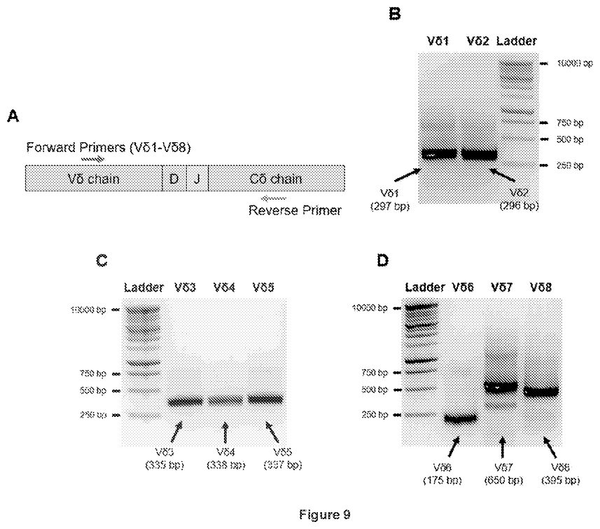

### Example 11: Successful Generation of a Full-Spectrum of Human γδ (Vδ1-Vδ8) T Cells Using the Diff-T Culture Method (FIG. 9)

Week 6 Diff-T cell culture was stimulated with 1 μg/ml anti-CD3/CD28 for 3 days in the presence of irradiated healthy donor PBMCs as antigen presenting cells in Diff-T M3 medium supplemented with 10 ng/ml IL-7 and 10 ng/ml IL-15. 3 days after stimulation, Diff-T cell culture was washed and cultured without anti-CD3/CD28 for additional 11 days. RNA extraction and RT-PCR were then performed to detect the successfully rearranged TCR Vδ1-Vδ8 chains utilized by different subsets of γδ T cells.

### Example 2: Ex Vivo Generation of HSC-Differentiated Human Diff-γδT and Diff-Tc Cells Engineered With Car and/or Other Immune Regulatory Genes

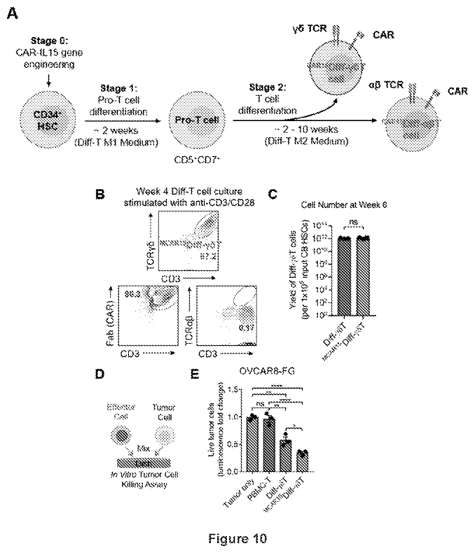

Generation of CAR-IL15-γδT cells (^{CAR15}Diff-γδT) by combining Diff-T culture method with CAR gene engineering (FIG. 10). Human CD34⁺ HSCs were engineered with genes encoding disease targeting molecules (e.g., chimeric antigen receptor (CAR)) and/or other immune regulatory genes (e.g., IL-15), then put into the Diff-T culture. The resulting Diff-γδT cells are expected to co-express the designated transgenes.

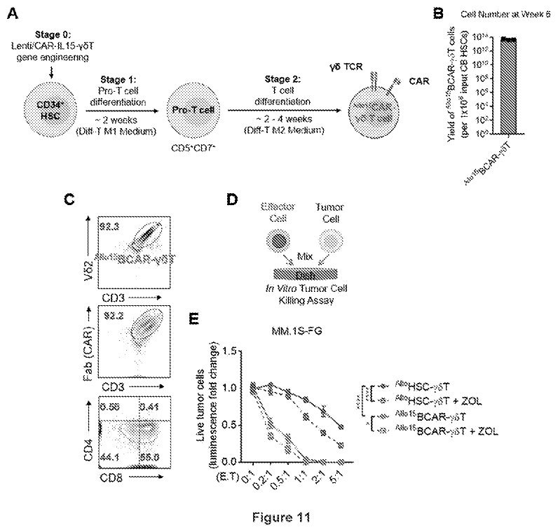

### Example 3: Generation of Allogeneic CAR-IL15-18T Cells (^{AlloCAR15}Diff-γδT) by Combining the Diff-T Culture Method With γδT TCR, CAR, and IL-15 Gene Engineering (FIG. 11)

Gamma delta T (γδ T) cells are a small subpopulation of T lymphocytes with the ability to bridge innate and adaptive immunity. The majority of γδ T cells in adult human blood exhibit Vγ9Vδ2 TCR and respond to small phosphorylated nonpeptide antigens, called phosphoantigens (pAgs), which are commonly produced by malignant cells. Unlike conventional αβ T cells, γδ T cells do not recognize the polymorphic classical major histocompatibility complex (MHC) molecules and are therefore free of GvHD risk when adoptively transferred into allogeneic host. Additionally, γδ T cells have several other unique features that make them ideal cellular carriers for developing off-the-shelf cellular therapy for cancer: 1) they have roles in cancer immunesurveillance; 2) they have the remarkable capacity to target tumors independent of tumor antigen-and major histocompatibility complex (MHC)-restrictions; 3) they can employ multiple mechanisms to attack tumor cells through direct killing and adjuvant effects; 4) they express a surface receptor, FcγRIII (CD16), that is involved in antibody-dependent cellular cytotoxicity (ADCC) and can be potentially combined with monoclonal antibody for cancer therapy. However, the development of an allogeneic off-the-shelf γδ T cellular product is greatly hindered by their availability—these cells are of extremely low number and high variability in humans (~1-5% T cells in human blood), making it very difficult to produce therapeutic numbers of γδ T cells from blood cells of allogeneic human donors.

The current method of generating γδ T cells, in particular the Vγ9Vδ2 subset, for adoptive therapy involves either ex vivo or in vivo expansion of peripheral blood mononuclear cell (PBMC)-derived γδ T cells using aminobisphosphonates, such as Zoledronate (ZOL). However, this method generates highly variable yields of γδ T cells depending on PBMC donors; and most importantly, such a γδ T cell product will likely contain bystander αβ T cells and thereby incurring GvHD risk. A novel method that can reliably generate a homogenous monoclonal population of γδ T cells at large quantities with a feeder-free differentiation system is thus pivotal to developing an off-the-shelf γδ T cell therapy.

CD34⁺ HSCs isolated from G-CSF-mobilized peripheral blood (denoted as PBSCs), or cord blood (denoted as CB HSCs) were transduced with a Lenti/γδT vector encoding a human γδT TCR gene then put into the Diff-T culture to generate ^{Allo}Diff-γδT cells. Both PBSCs and CB HSCs can effectively differentiate into and expand as transgenic ^{Allo}Diff-γδT cells. Similarly, ^{AlloCAR}Diff-γδT cells can be generated by transducing the HSCs with a lentiviral vector encoding a human γδT TCR gene together with a CAR gene. In addition, ^{AlloCAR15}Diff-γδT cells can be generated by transducing the HSCs with a lentiviral vector encoding a human γδT TCR gene together with a CAR gene and an IL-15 gene (FIGS. 11A, 11B, and 11C). It is estimated that ~10¹³ scale of ^{AlloCAR15}Diff-γδT cells can be produced from either PBSCs of a healthy donor or HSCs of a CB sample, which can be formulated into 10,000-100,000 doses (at ~10⁸-10⁹ cells per dose) (FIG. 11B). Unless otherwise indicated, CB HSC-derived ^{Allo}Diff-γδT cells and their derivatives such as ^{AlloCAR15}Diff-γδT cells were utilized for the proof-of-principle studies described below.

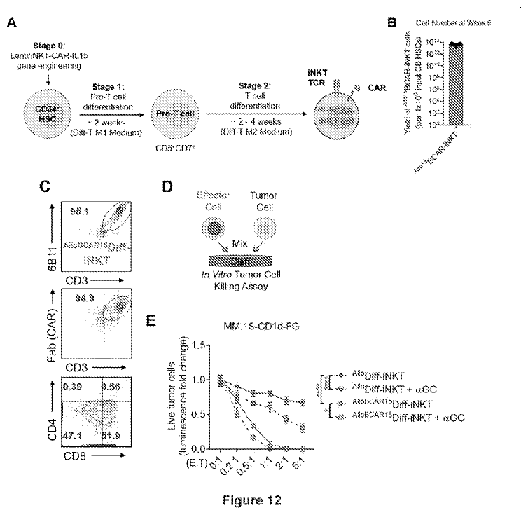

### Example 4: Generation of Allogeneic CAR-IL15-iNKT Cells (^{AlloCAR15}Diff-iNKT) by Combining the Diff-T Culture Method With iNKT TCR, CAR, and IL-15 Gene Engineering (FIG. 12)

Invariant natural killer T (iNKT) cells are a small subpopulation of αβ T lymphocytes with the ability to bridge innate and adaptive immunity. Unlike the conventional αβ T cells, the T cell receptor (TCR) of iNKT cells recognizes lipid antigens presented by CD1d, a major histocompatibility complex (MHC)-like molecule, instead of MHC itself. Because of this unique property, iNKT cells do not cause graft-versus-host disease (GvHD) when transplanted allogeneically. Additionally, iNKT cells have several other unique features that make them ideal cellular carriers for developing off-the-shelf cellular therapy for cancer: 1) they have roles in cancer immunesurveillance; 2) they have the remarkable capacity to target tumors independent of tumor antigen-and major histocompatibility complex (MHC)-restrictions; 3) they can employ multiple mechanisms to attack tumor cells through direct killing and adjuvant effects. However, the development of an allogeneic off-the-shelf iNKT cellular product is greatly hindered by their availability—these cells are of extremely low number and high variability in humans (~0.001-1% in human blood), making it very difficult to produce therapeutic numbers of iNKT cells from blood cells of allogeneic human donors.

Two prior methods have been used to generate enough iNKT cells for therapeutic uses. One method is to screen large numbers of donors and find “super donors” who naturally have high percentage of iNKT cells in peripheral blood. iNKT cells are enriched by the magnetic bead-based purification procedure and then expanded by either anti-CD3/CD28 bead stimulation or co-culture with antigen-presenting cells loaded with alpha-galactosylceramide (αGC). Although expansion can be achieved by this method, the expansion fold is limited, and the expansion is unreliable. Another method is based on the genetic modification of hematopoietic stem cells (HSCs) with iNKT TCRs followed by an artificial thymic organoid (ATO) culture system that supports the ex vivo differentiation of human HSCs into iNKT cells. Although this method can generate iNKT cells with high yield, the production requires the use of feeder cells of mouse origin, which poses significant challenges to develop a reliable process for GMP-compatible manufacturing.

A novel method that can reliably generate a homogenous monoclonal population of iNKT cells at large quantities with a feeder-free differentiation system is thus pivotal to developing an off-the-shelf iNKT cell therapy.

CD34⁺ HSCs isolated from G-CSF-mobilized peripheral blood (denoted as PBSCs), or cord blood (denoted as CB HSCs) were transduced with a Lenti/iNKT vector encoding a human iNKT TCR gene then put into the Diff-T culture to generate ^{Allo}Diff-iNKT cells. Both PBSCs and CB HSCs can effectively differentiate into and expand as transgenic ^{Allo}Diff-iNKT cells. Similarly, ^{AlloCAR}Diff-iNKT cells can be generated by transducing the HSCs with a lentiviral vector encoding a human iNKT TCR gene together with a CAR gene. In addition, ^{AlloCAR15}Diff-iNKT cells can be generated by transducing the HSCs with a lentiviral vector encoding a human iNKT TCR gene together with a CAR gene and an IL-15 gene (FIGS. 12A, 12B, and 12C). It is estimated that ~10¹³ scale of ^{AlloCAR15}Diff-iNKT cells can be produced from either PBSCs of a healthy donor or HSCs of a CB sample, which can be formulated into 10,000-100,000 doses (at ~10⁸-10⁹ cells per dose) (FIG. 12B). Unless otherwise indicated, CB HSC-derived ^{Allo}Diff-iNKT cells and their derivatives such as ^{AlloCAR15}Diff-iNKT cells were utilized for the proof-of-principle studies described below.

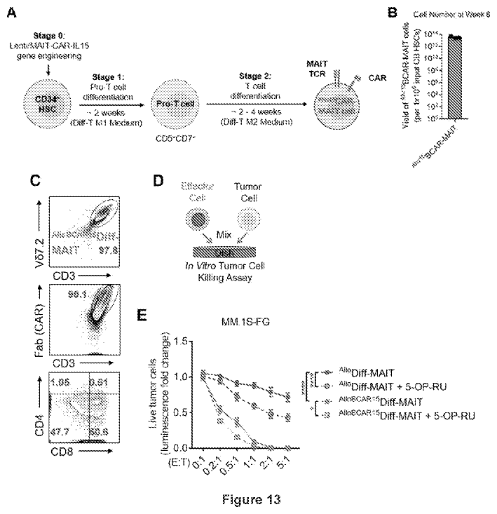

### Example 5: Generation of Allogeneic CAR-IL15-MAIT Cells (^{AlloCAR15}Diff-MAIT) by Combining the Diff-T Culture Method With MAIT TCR, CAR, AND IL-15 Gene Engineering (FIG. 13)

Human mucosal-associated invariant T (MAIT) cells, an evolutionarily conserved, innate-like subset of T cells, currently stand as an untapped frontier that holds immense potential as a cutting-edge cancer immunotherapy. As suggested by their name, MAIT cells primarily localize to mucosa rich regions, comprising a notable fraction of T cells distributed throughout the pulmonary (5%), hepatic (20-40%), and intestinal (1-2%) lamina propria, as well as peripheral circulation (1-10%). Given their innate-like quality, MAIT cells display a heavily restricted TCR repertoire, which has been canonically defined as expression of an invariant TCRα chain Vα7.2-Jα33 paired with a limited number of TCR β chains, predominantly Vβ2/Vβ13. These TCRs recognize MHC-related molecule 1 (MR1), a non-classical, MHC Class I-like molecule that loads riboflavin-derived metabolites synthesized by bacteria to activate quiescent MAIT cells. Owing to their semi-invariant TCRs, MAIT cells do not induce GvHD upon allogeneic cell transfer. Multiple in vitro studies have demonstrated that MAIT cells exhibit solid tumor-infiltrating capacity and direct anti-tumor cytotoxicity. These characteristics suggest MAIT cells as a promising candidate for developing “off-the-shelf” cell therapy. MAIT cells only account for 1-8% of circulating T cells in healthy individuals, and thus there is an unmet need for large-scale manufacturing of MAIT cells with enhanced anti-tumor efficacy. By transducing MAIT TCR into CD34+ HSCs and culturing these HSCs with our established Diff-T culture method, we can generate allogeneic HSC-derived MAIT cells for off-the-shelf immunotherapy.

CD34⁺ HSCs isolated from G-CSF-mobilized peripheral blood (denoted as PBSCs), or cord blood (denoted as CB HSCs) were transduced with a Lenti/MAIT vector encoding a human MAIT TCR gene then put into the Diff-T culture to generate ^{Allo}Diff-MAIT cells. Both PBSCs and CB HSCs can effectively differentiate into and expand as transgenic ^{Allo}Diff-MAIT cells. Similarly, ^{AlloCAR}Diff-MAIT cells can be generated by transducing the HSCs with a lentiviral vector encoding a human MAIT TCR gene together with a CAR gene (FIG. 13A). In addition, ^{AlloCAR15}Diff-MAIT cells can be generated by transducing the HSCs with a lentiviral vector encoding a human MAIT TCR gene together with a CAR gene and an IL-15 gene (FIGS. 13B and 13C). It is estimated that ~10¹³ scale of ^{AlloCAR15}Diff-MAIT cells can be produced from either PBSCs of a healthy donor or HSCs of a CB sample, which can be formulated into 10,000-100,000 doses (at ~10⁸-10⁹ cells per dose) (FIG. 13B). Despite the differences in expansion fold, ^{Allo}Diff-MAIT cells and their derivatives generated from PBSCs, and CB HSCs displayed similar phenotype and functionality. Unless otherwise indicated, CB HSC-derived ^{Allo}Diff-MAIT cells and their derivatives such as ^{AlloCAR15}Diff-MAIT cells were utilized for the proof-of-principle studies described below.

All publications mentioned herein (e.g., U.S. Patent Publications 20220257655 and 20210123022, and the references numerically listed herein) are incorporated herein by reference to disclose and describe aspects, methods and/or materials in connection with the cited publications.

### REFERENCES

1. Themeli, M., et al., Generation of tumor-targeted human T lymphocytes from induced pluripotent stem cells for cancer therapy. Nat Biotechnol, 2013. 31(10): p. 928-33.
2. Seet, C. S., et al., Generation of mature T cells from human hematopoietic stem and progenitor cells in artificial thymic organoids. Nat Methods, 2017. 14(5): p. 521-530.
3. Montel-Hagen, A., et al., Organoid-Induced Differentiation of Conventional T Cells from Human Pluripotent Stem Cells. Cell Stem Cell, 2019. 24(3): p. 376-389 e8.
4. Wang, B., et al., Generation of hypoimmunogenic T cells from genetically engineered allogeneic human induced pluripotent stem cells. Nat Biomed Eng, 2021. 5(5): p. 429-440.
5. Shukla, S., et al., Progenitor T-cell differentiation from hematopoietic stem cells using Delta-like-4 and VCAM-1. Nat Methods, 2017. 14(5): p. 531-538.
6. Iriguchi, S., et al., A clinically applicable and scalable method to regenerate T-cells from iPSCs for off-the-shelf T-cell immunotherapy. Nat Commun, 2021. 12(1): p. 430.
7. Jing, R., et al., EZH1 repression generates mature iPSC-derived CAR T cells with enhanced antitumor activity. Cell Stem Cell, 2022. 29(8): p. 1181-1196 e6.
8. Michaels, Y. S., et al., DLL4 and VCAM1 enhance the emergence of T cell-competent hematopoietic progenitors from human pluripotent stem cells. Sci Adv, 2022. 8(34): p. eabn5522.
9. Manning, J., et al., Vitamin C promotes maturation of T-cells. Antioxid Redox Signal, 2013. 19(17): p. 2054-67.

1. A method of making Diff-T cells using hematopoietic stem and progenitor cells (HSPCs or HSCs) that comprises, without or with introducing into Diff-T cells, a selected monoclonal T cell receptor (TCR) gene:
(a) without a selected monoclonal TCR gene, endogenous TCRs arising from TCR recombination during HSC to T cell differentiation is used to make the Diff-T cells; or
(b) with a selected monoclonal TCR gene, an exogeneous TCR transgene is introduced to HSCs to make the Diff-T cells; and
differentiating the HSCs of (a) or (b) so as to generate the Diff-T cells.

2. The method of claim 1, wherein the HSCs can come from but not limited to:
umbilical cord blood (CB)
peripheral blood (PB)
pluripotent stem cells (PSCs)

3. The method of claim 1, wherein the endogenous TCRs arising from TCR recombination during HSC to T cell differentiation can be but not limited to:
an alpha beta TCR (a conventional CD4 αβ TCR, a conventional CD8 αβ TCR, or an unconventional αβ TCR),
a gamma delta TCR (a Vδ1 TCR, a Vδ2 TCR, a Vδ3 TCR, or other γδ TCRs),
an invariant NKT (iNKT) TCR,
a non-invariant NKT TCR, and
a mucosal associated invariant T (MAIT) TCR.

4. The method of claim 1, wherein the monoclonal TCR gene encodes a TCR selected from but not limited to:
an alpha beta TCR (a conventional CD4 αβ TCR, a conventional CD8 αβ TCR, or an unconventional αβ TCR),
a gamma delta TCR (a Vδ1 TCR, a Vδ2 TCR, a Vδ3 TCR, or other γδ TCRs),
an invariant NKT (iNKT) TCR,
a non-invariant NKT TCR, and
a mucosal associated invariant T (MAIT) TCR.

5. The method of claim 1, wherein a Diff-T cell product comprises a gene expression profile characterized as being at least one of:
polyclonal TCR-positive CD3-postive,
monoclonal TCR-positive CD3-postive,
expression of one or more immune modulatory transgene(s), and/or
disrupted expression of one or more endogenous immune modulatory gene(s).

6. The method of claim 5, wherein
(a) the immune regulatory transgene(s) delivered into a Diff-T cell product can encode any one or more of the following: immune targeting molecules (e.g., chimeric antigen receptors, CARs; T-cell receptors, TCRs; native or synthetic receptor/ligands, and others), immune regulatory molecules (e.g., IL-2, IL-4, IL-7, IL-10, IL-12, IL-15, IL-18, IL-21, IFN-γ, TNF-α, TL1A, CD27, CD28, 4-1BB, OX40, ICOS, DAP10, Bcl11b, Batf3, ThPOK, FOXP3, Runx3, and others), immune allorejection resistance molecules (e.g., HLA-C, HLA-E, HLA-G, CD47, and others), and/or suicide control and imaging marker molecules (e.g., sr39TK, iCasp9, CD20, and others); and
(b) the endogenous gene(s) disrupted in the Diff-T cells can encode any of the following: immune checkpoint molecules (e.g., PD-1, PD-L1, CTLA-4, LAG-3, TIM-3, TIGIT, B7-H3/B7-H4, BTLA, VISTA, NKG2A, A2aR, PVRIG, IDO, CD73, CD39, CD96, CD161, and others), immune regulatory molecules (e.g., TET2, PI3Kδ/γ, DGK, DNMT3a, Suv39h1, and others), or/and immune allorejection molecules (e.g., HLA-I/II, B2M, CIITA, and others).

7. The method of claim 1, wherein all the desired gene modifications intended for a designated Diff-T cell product are introduced to HSCs with:
(a) a single transgene or multiple transgenes. The transgenes can be incorporated into a Diff-T cell product via any of a wide variety of gene delivery vectors (e.g., lentivector, retrovector, adenovector, AAV, and others) and/or vector-free systems (e.g., CRISPR, TALEN, Zinc-Finger, and others), and/or
(b) a single endogenous gene or multiple endogenous genes of a Diff-T cell product can be disrupted from expression via any of a wide variety of gene editing tools (e.g., CRISPR, TALEN, Zinc-Finger, and others).

8. The method of claim 1, wherein HSCs are cultured ex vivo to produce Diff-T cells through 4 stages and certain optional steps:
Stage 0 that supports the genetic modification of HSCs, and
Stage 1 that supports the HSC differentiation into progenitor-T (pro-T) cells, and
Stage 2 that supports the pro-T cell differentiation into mature Diff-T cells, and
Stage 3 that supports the expansion of Diff-T cells, and
an optional “CD4-Induction Step” that can be added between the Stage 2 and Stage 3 cultures to enable the generation of CD4 single-positive (CD4 SP) Diff-T cells, and
another optional “T_{H}-Polarization Step” that can be further added in Stage 3 culture to enable the generation of T_{H}-polarized CD4 SP Diff-T cells.

9. The method of claim 8, wherein:
all 3 culture stages (Stages 1, 2, and 3) can be feeder-free and/or serum-free; or
the Stage 3 culture can contain feeder cells (e.g., artificial antigen presenting cells; APCs); and/or
the cell culture media can comprise a base medium supplemented with one or more factors selected to facilitate the HSC-derived Diff-T cell differentiation, expansion, and sublineage commitment; and/or
all three stages (Stages 1, 2, and 3) of ex vivo culture can achieve high purity, eliminating the need for in-process purification steps; and/or
the HSC-derived Diff-T intermediate cell product(s) can be cultured freshly or cryopreserved and then thawed for continued culture.

10. The method of claim 1, wherein:
a Diff-T cell product produced by the methods described herein can be cryopreserved; and/or
the cryo-recovered cell product can be stable at room temperature for at least one hour; and/or
the cryo-recovered cell product is stable at room temperature for at least 1, 2, 3, 4, 5, 6, 7, 8, 10, 15, 20, 24, 30, or 48 hours (or any derivable range therein); and/or
a cell product contains a solution comprising dextrose, one or more electrolytes, albumin, dextran, and/or DMSO; and/or
a cell product is in a solution that is sterile, nonpyogenic, and isotonic.

11. The method of claim 1, wherein a Diff-T cell product can be used for treating patients:
the patient has a cancer; and/or
the patient has a viral, bacterial, fungal or parasitic infection; and/or
the patient has a disease or condition involving inflammation, which, in some embodiments, excludes cancer; and/or
the patient has an autoimmune disease or condition; and/or
the Diff-T cell product is allogeneic with respect to the patient; and/or
the patient does not exhibit signs of rejection or depletion of the Diff-T cells; and/or some therapeutic methods further include administering to the patient a stimulatory reagent that activates Diff-T cells, or a reagent that triggers the suicide gene kill-switch.

12. The method of claim 1, wherein the method comprises:
(a) collecting and/or enriching CD34⁺ HSCs obtained from umbilical cord blood, peripheral blood, and PSC-derived HSCs;
(b) with or without genetic modifying CD34⁺ HSCs obtained from (a);
(c) disposing the CD34⁺ HSCs obtained from (b) in a serum-free, feeder free Diff-T M1 cell culture media comprising at least one of serum albumin, recombinant human insulin, human transferrin, human LDL, 2-mercaptoethanol, L-ascorbic acid, SCF, TPO, IL-7, IL-3, IL-6, Flt 3 ligand, SDF- 1α, and StemRegenin 1 for at least 14 days; and
(c) disposing the pro-T cells obtained from (b) in a serum-free, feeder free Diff-T M2 cell culture media comprising at least one of serum albumin, recombinant human insulin, human transferrin, human LDL, 2-mercaptoethanol, L-ascorbic acid, SCF, IL-7, Flt3 ligand, SDF-1α, StemRegenin 1, IL-2, IL-21, IL-15, TGF-β;
such that the Diff-T cells are made.

13. The method of claim 12, further comprising disposing the Diff-T cells into a Diff-T M3 cell culture media comprising at least one of serum albumin, one or multiple of supporting cytokines and/or small molecules and additives, one of a TCR cognate antigen, or a non-specific TCR stimulatory reagent.

14. The method of claim 1, wherein the exogenous nucleic acid molecule encodes at least one T cell receptor selected from:
an alpha beta TCR;
a gamma delta TCR,
an invariant NKT (TARGET) TCR,
a non-invariant NKT TCR, and
a mucosal associated invariant TCR.

15. The method of claim 1, wherein;
the exogenous nucleic acid molecule is contained in a lentiviral expression vector; and/or
the exogenous nucleic acid molecule further encodes a polypeptide that stimulates T cells, a polypeptide that disrupts T cell inhibitory factors, and/or a polypeptide comprising a further receptor.

16. The method of claim 15, wherein the polypeptide comprises at least one of a chimeric antigen receptor (CAR), IL-2, IL-7, IL-10, IL-12, IL-15, membrane-bound IL-15, IL-18, TL1A, IFN-γ, TNF-α, CD28, 4-1BB, CD27, OX40, ICOS, and FOXP3.

17. The method of claim 13, wherein Diff-T M3 cell culture media may comprise feeder cells.

18. The method of claim 13, wherein Diff-T M3 cell culture media comprises at least one of IL-2, IL-7 and IL-15.

19. The Diff-T cells of claim 1, wherein the Diff-T cells express CD4.
